(19) United States
(12) Patent Application Publication
(10) Pub. No.: US 2025/0145962 A1
(43) Pub. Date: May 8, 2025

(54) COMPOSITIONS, SYSTEMS, AND METHODS RELATING TO THREE-DIMENSIONAL (3D) BIOPRINTED LIVER TISSUE MODELS

(71) Applicants: UNIVERSITY OF FLORIDA RESEARCH FOUNDATION, Gainesville, FL (US); MERCK SHARP & DOHME CORP., Rahway, NJ (US)

(72) Inventors: Thomas Ettor ANGELINI, Gainesville, FL (US); Carolina Elizabeth ABRAHAN, Gainesville, FL (US); Steven CHISOLM, Gainesville, FL (US); Jon P. DOBSON, Gainesville, FL (US); Clayton Elwood MATHEWS, Gainesville, FL (US); Peter S. MCFETRIDGE, Gainesville, FL (US); Glyn Daniel PALMER, Gainesville, FL (US); Vignesh SUBRAMANIAM, Gainesville, FL (US); Brent S. SUMERLIN, Gainesville, FL (US); Andreas BAUDY, Gainesville, FL (US); Brett HIGGINS, Gainesville, FL (US)

(21) Appl. No.: 18/695,063

(22) PCT Filed: Oct. 4, 2022
(86) PCT No.: PCT/US2022/077536

§ 371 Date: Mar. 25, 2024

Related U.S. Application Data
(60) Provisional application No. 63/252,334, filed on Oct. 5, 2021.
(60) Provisional application No. 63/272,893, filed on Oct. 28, 2021.

(51) Int. Cl. C12N 5/071; C12M 1/00; C12M 3/00

(52) U.S. Cl. CPC C12N 5/0671; C12M 21/08; C12M 29/10; C12N 5/069; C12N 2503/02; C12N 2533/54

(57) ABSTRACT

Disclosed herein are compositions, systems, and methods relating to three-dimensional bioprinted tissue models, in particular liver tissue modes. Compositions, systems, and methods as described here provide for improved tissue models, thereby improving their utility for diagnostic, clinical, and research purposes. In certain aspects, tissue models as described herein exhibit gene expression profiles, enzymatic function, and enzyme/protein secretion comparable to in vivo organs.

### CROSS-REFERENCE TO RELATED APPLICATIONS

This application claims benefit of U.S. Provisional Application No. 63/252,334, filed Oct. 5, 2021, and U.S. Provisional Application No. 63/272,893, filed Oct. 28, 2021, which are hereby incorporated herein by reference in their entireties.

### STATEMENT REGARDING FEDERALLY SPONSORED RESEARCH OR DEVELOPMENT

This invention was made with government support under Grant No. W911NF-17-3-0003 awarded by the United States Department of Defense. The government has certain rights in the invention.

### BACKGROUND

Animal models and in vitro cell culture approaches are widely used to test compounds for potential liver toxicity during the drug development process. However, numerous limitations of both general approaches are widely recognized; animal models often fail to predict toxicity in humans while in vitro models lack the complexity of real tissue that is required for proper function and the related response to compounds. Thus, to increase the pace and lower the costs of drug development, there is a critical need for technologies capable of predicting the toxicity of compounds.

Accordingly, there is a need to address the aforementioned deficiencies and inadequacies to provide for improved devices, systems, and methods related to improved tissue models.

### SUMMARY

Disclosed herein are planar cellular compositions. In certain aspects, planar cellular compositions as described herein comprise hepatocytes and endothelial cells. In certain aspects, the hepatocytes and endothelial cells can be primary cells.

Disclosed herein is a planar cellular composition, comprising hepatocytes and endothelial cells in a matrix material, wherein the hepatocytes and endothelial cells are present in a ratio of about 2:1 to 1:1 hepatocytes:endothelial cells. In some embodiments, the matrix material comprises collagen.

Also disclosed herein is a method of creating a planar cellular composition that involves mixing hepatocytes, endothelial cells, and a matrix material in a cellular growth medium to form a cellular mixture, wherein the hepatocytes and endothelial cells are present in a ratio of about 2:1 to 1:1 hepatocytes:endothelial cells; placing the cellular mixture in a support medium to form a planar cellular composition about 5 to 20 cells in thickness and about 0.5 to 10 mm diameter; and culturing the cellular mixture under physiological growth conditions to produce the planar cellular composition. In some embodiments, the hepatocytes are primary cells. In some embodiments, the endothelial cells are vascular endothelial cells. In some embodiments, the cellular mixture further contains cholangiocytes.

In some embodiments, the support material comprises polymeric packed microgel particles. In some embodiments, the support material comprises poly-ethylene glycol (PEG) microgels.

In some embodiments, the cellular growth medium comprises supplements to facilitate hepatocyte viability/function, but do not contain supplements that promote angiogenesis. For example, in some embodiments, the cellular growth medium comprises hepatocyte growth factor (HGF), Y27632 (Rock inhibitor), A83-01 (TGF-b inhibitor), and/or CHIR99021 (GSK3 inhibitor), but does not contain vascular endothelial growth factor (VEGF). Hepatocyte Growth Factor (HGF), Y27632 (Rock inhibitor), A83-01 (TGF-b inhibitor) and CHIR99021 (GSK3 inhibitor).

In some embodiments, the placing is performed with a three-dimensional (3D) printer. In some embodiments, the placing is in a spiral pattern extending radially and annularly around an initial placement point.

Also disclosed herein is a planar cellular composition produced according to a method disclosed herein.

Also disclosed herein is a tissue culture system that involves a planar cellular composition disclosed herein embedded in a support medium; and a bioreactor. In some embodiments, the system further includes a cellular growth medium. In some embodiments, the cellular growth medium comprises hepatocyte growth factor (HGF) but does not comprise vascular endothelial growth factor (VEGF). In some embodiments, the bioreactor is a perfusion-enabled bioreactor. In some embodiments, the support medium comprises polymeric packed microgel particles. In some embodiments, the support medium comprises poly-ethylene glycol (PEG) microparticles.

Also disclosed herein is a method for screening candidate agents, comprising contacting the candidate agent to a planar cellular composition disclosed herein and evaluating one or more properties of the planar cellular composition. In some embodiments, the candidate agent is a therapeutic candidate selected from the group consisting of a small molecule, a protein, and a nucleic acid. In some embodiments, the candidate agent is a toxicity candidate selected from the group consisting of a small molecule, a protein, a nucleic acid, and a pathogen. In some embodiments, the one or more properties involves albumin/urea synthesis rates, cell viability/morphology, cell death/apoptosis, ADME gene expression and function, or a combination thereof.

In certain aspects, the hepatocytes and endothelial cells can be present in a ratio of about 2:1 hepatocytes:endothelial cells. In certain aspects, the planar cellular composition can have at least one physical dimension between about 20 and 200 microns in size. In certain aspects, the planar cellular composition can have at least one physical dimension of two to ten cells in size. In certain aspects, the planar composition can have a circular or oval shape. In certain aspects, the circular or oval shape has a about 0.5 to 10 mm diameter, including about 1.5 to 2.5 mm in diameter. In some embodiments, the endothelial cells form one or more sinusoidal structures that are not organized into a network.

Described herein are methods of making a cellular composition. In certain aspects, a method of creating a planar cellular composition, comprises mixing hepatocytes, endothelial cells, and a matrix material in a medium to form a cellular mixture, and placing the cellular mixture in a support medium to form a planar cellular composition.

In certain aspects, the hepatocytes and endothelial cells can be primary cells. In certain aspects, the endothelial cells are vascular endothelial cells. In certain aspects, the hepatocytes and endothelial cells are present in a ratio of about 2:1 hepatocytes:endothelial cells. In certain aspects, the planar cellular composition can have at least one physical dimension between about 20 and 200 microns in size. In some embodiments, the planar cellular composition has at least one physical dimension between about 5 to 20 cells in thickness, including 2 to 10 cells in size. In certain aspects, the planar composition can have a circular or oval shape. In certain aspects, the matrix can be reconstituted liver matrix, Matrigel®, or a combination thereof. In certain aspects, the support material can comprise hydrogel particles. In certain aspects, the support material can comprise poly-ethylene glycol (PEG) microgels. In certain aspects, the placing can be performed with a three-dimensional (3D) printer. In certain aspects, the placing can be in a spiral pattern extending radially and annularly around an initial placement point.

Also described herein are tissue culture systems. In certain aspects, a tissue culture system, comprises a planar cellular composition as described herein embedded in a support medium and a bioreactor. In certain aspects, tissue culture systems as described herein can further comprise a cellular growth medium. In certain aspects, the bioreactor can be a perfusion-enabled bioreactor. In certain aspects, the support medium can comprise polymeric packed microgel particles. In certain aspects, the support medium can comprise poly-ethylene glycol (PEG) microparticles.

Also described herein are methods of using a tissue culture system. In certain aspects, a method of using a tissue culture system can comprise providing a tissue culture system as described herein and providing cellular growth medium to the planar cellular composition. In certain aspects, methods of using a tissue culture system as described herein can further comprise providing a therapeutic candidate or toxicity candidate to the planar cellular composition. In certain aspects, a therapeutic candidate can comprise one or more of a small molecule, protein, or nucleic acid. In certain aspects, the toxicity candidate can comprise one or more of a small molecule, protein, nucleic acid, bacterium, or virus.

The details of one or more embodiments of the invention are set forth in the accompanying drawings and the description below. Other features, objects, and advantages of the invention will be apparent from the description and drawings, and from the claims.

### DESCRIPTION OF DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIGS. 1A and 1B show albumin (FIG. 1A) and urea (FIG. 1B) production of tissue models as described herein. The February experiment met the minimum albumin production and urea synthesis rates (shown by dotted line) and stability between Day 13-20 was observed within the same run. Exp 1 and Exp 2 were conducted separately at shorter timeframes and with a separate human donor (#2005) for Exp 2.

FIGS. 2A and 2B show albumin (FIG. 1A) and urea (FIG. 1B) production of tissue models as described herein. An experiment evaluating the printed UF model using hepatocyte donor #1980 exceeded the minimum albumin production and urea synthesis rates (shown by dotted line) and a number of stable time ranges were observed. The inter-experiment variability between March and February experiments (both #1980 donor) is also shown.

FIGS. 3A-3L depict enzymatic functional analysis of tissue models as described herein. Enzyme functional analysis was performed in 2 independent experiments, the first of which in March demonstrated that the expected metabolites produced from incubated parent compound were all detected. The formation rates of these metabolites was linear and the SW in-well incubation condition selected, compared to incubations performed with tissues that were extracted from printed wells (SE). The follow-up April experiment used the SW condition and compared the metabolite formation rates from 2 different hepatocyte donors in the 3D printed model compared to a primary hepatocyte suspension control.

FIGS. 4A-4K3: Theoretical mRNA copy numbers measured are shown and compared between groups, including human hepatocyte donors (green: sourced from Agilent and Thermo Fisher), human HepatoPac (red: at DO and D9), and the 3D printed human liver (using the same donor source as previous experiments, Donor #1980) with one group being Static (blue: at D14 and D21), and a separate group comparing Perfusion with Static (blue: at D14 and D21). FIG. 4A shows the levels of a set of hepatocyte endogenous control genes, while FIGS. 4B-4K3 show the data for the 56 ADME genes that are part of the Stage 1 criteria, categorized by type.

FIG. 5 is a schematic of an embodiment of precipitation polymerization of charged copolymer microgels; micrographs of microgels were collected to confirm proper synthesis and estimate average particle size.

FIG. 6 is a schematic of inverse emulsion polymerization of uncharged microgels; micrographs of microgels were collected to confirm proper synthesis and estimate average particle size. These particles were found to be nearly perfect spheres, in contrast to microgels synthesized by precipitation polymerization.

FIG. 7 shows chemical structures of charged monomers and schematic of synthesis of charged PEG microgels.

FIG. 8 shows micrographs and data visualization of live dead assays. Live dead assays demonstrate that the spherical microgels synthesized using inverse emulsion are significantly less toxic than those synthesized using precipitation polymerization. Charge-neutral PEG microgels always perform better than charged microgels.

FIG. 9 is a NMR spectra of qDMAEMA.

FIG. 10 shows needle uncertainty and errors in needle positioning between moves. The uncertainty in locating the needle tip was determined by mounting the DISC printer atop an inverted fluorescence microscope. By analyzing images of the needle tip (upper left), extremely low uncertainties in repeatedly locating the needle center were found. By moving the needle in a square 1 mm×1 mm path and measuring the needle location relative to the programmed location, we measured the DISC 3D bioprinter positioning error. The positioning errors to be less than 10 μm on average, never reaching 20 μm.

FIG. 11 depicts bioprinting workflow. Tissues were 3D printed using the DISC bioprinting instrument mounted atop an inverted fluorescence microscope (left). The DISC system has the capability to locate the needle tip with a crossed laser-sensor system and accommodates well-plates. Following printing, the well plates containing the 3D printed tissues are transferred to an inverted confocal microscope for imaging (right).

FIG. 12 depicts an embodiment of a biofabrication approach. Tissues were printed in spiral paths to create tissues in a single smooth motion, reducing the appearance of unwanted features. The feature width is matched to the spiral pitch and the resulting tissue is 200 μm thick. This strategy as employed using several different hepatocyte sources.

FIG. 13 depicts aspects of bioprinted tissue metrology. The effective tissue diameter is determined by performing maximum-intensity projections of 3D confocal stacks along the thin-axis of the disk-shaped structures, then thresholding. The area of the thresholded structures was measured and converted to a diameter that accounts for the asymmetric spiral shape and the feature width.

FIG. 14 is a graft depicting measured vs. expected diameter. The measured diameters of the structures, dm, correlated extremely well with the expected diameters, de. The standard deviation of repeated measurements and the average error in absolute size grow with increasing de. Overall, these data show that the tissues are within the specified variabilities and absolute errors of their diameters as proposed.

FIG. 15 show cellular deposition as visualized with fluorescence microscopy. To demonstrate the multi-material capabilities of the DISC printer system, a layer of hepatocytes and a layer of cholangiocytes was printed with a single programmed process. The cholangiocytes were first printed, and the hepatocytes were deposited at differing distances above the cholangiocytes. In the case where there was no separation, the two cell types mixed and were co-located in the layer; with increasing separation, the green and red fluorescence profiles were observed to move apart along the z-axis with well-controlled spacing.

FIGS. 16A-16B are plots showing quantitative measurements of albumin by ELISA (FIG. 16A) and urea by Urea Nitrogen (BUN) assay (FIG. 16B) in PEG microgel media.

FIGS. 17A-17B are plots showing the effects of increasing sample assay volume for enhanced detection of urea using the Urea Nitrogen BUN assay. FIG. 17A Curve fit and regression analysis for standard urea concentrations. FIG. 17B Curve fit of replotted data in (a), marked by grey box, to reveal enhanced sensitivity of low urea concentrations with increased sample volumes.

FIGS. 18A-18C show distribution of albumin and urea secreted by hepatocyte cell cultures in PEG microgel and media supernatants.

FIGS. 19A-19D are plots showing Determination of cell number in 3D printed cell constructs cultured in microgel media. Calibration plots generated by PicoGreen assay using known DNA standards (FIG. 19A) or cell concentrations (FIG. 19B). Cell calibration for MG63 (10³-10⁶ cells) is shown in b. FIG. 19C:Cell number determination of printed MG63 1.5 mm spheroids cultured in pAAm or PEG. FIG. 19D: HepaRG cell number from printed discs (2 mm d) recovered from PEG microgels subjected to perfusion or static flow culture.

FIG. 20 is a cartoon schematic showing construction of pAlb and pEAlb gene reporter vectors. The proximal promoter, with and without the enhancer region of the human albumin gene were cloned into dual expression lentiviral vectors. The promoter region is enriched for transcription factor binding sites that direct liver-specific expression and albumin synthesis.

FIGS. 21A-21H are data showing Activity of albumin gene reporter constructs in HepG2/C3A and MG63 cell lines. FIGS. 21A-21F: Merged red/green fluorescent images of cells labeled with pAlb and pEAlb reporter vectors. FIGS. 21G-21H: Frequency of GFP+/td-tom+ cells within each transduced cell population. 3 fields were counted for each group. Panels a,b,d,e: Mag.=4×; panels c, f: Mag.=20×.

FIG. 22 is a plot showing oxidized nifedipine formation.

FIG. 23 is a plot showing dextrorphan formation.

FIG. 24 is a plot showing hydroxydiclofenac formation.

FIG. 25 is a plot showing acetaminophen formation.

FIG. 26 is a plot showing hydroxybupropion formation.

FIG. 27 is a plot showing 7-hydroxyfoumarin glucuronide formation.

FIG. 28 show thiol-ene reaction mechanisms (radical mechanistic pathway) (top), and thiol-Michael addition (nucleophilic mechanistic pathway) (bottom) to result in the anti-Markovnikov thiol-ene product.

FIG. 29 is a synthetic scheme to prepare fluorescein-SH and the 1H NMR spectrum of the resulting product.

FIG. 30 show thiol-ene (top) and thiol-Micheal (bottom) addition synthetic outline for functionalization of PEG microgels with fluorescein thiol.

FIG. 31 is a plot of UV-Vis spectra of the fluorescein-SH starting material, the PEG microgels prior to reaction, and the fluorescein-PEG microgel after extensive dialysis. Appearance of the fluorescein absorption in the functionalized PEG microgels offers evidence of successful conjugation and confirms targeting of residual vinyl groups is a viable strategy for microgel derivatization.

FIG. 32 depicts a reaction scheme for thiol-Michael addition of Gelatin-SH to swollen PEG microgels in PBS.

FIG. 33 is an ATR-FTIR spectra of PEG microgels in the region of the residual vinyl group band near 2100 cm⁻¹. After conjugation of Gelatin-SH and fluorescein-thiol (Fluor-SH), this band disappears, suggesting consumption of the residual vinyl groups during the thiol-Michael addition.

FIG. 34 shows preparation of core-shell microgels from polymerization of NIPAM monomer in the presence of PEG microgels (top). Increased rigidity between gel particles after heating due to hydrophobically sphered connections between the PNIPAM shells of neighboring particles (bottom).

FIGS. 35A-B are microgel micrographs. FIG. 35A: False phase contrast overlay of PEG-NIPAM microgels at 25° C. and 50° C. FIG. 35B. Individual microscopic images of microgels at 25 and 50° C. Time indicates diffusion time of monomer prior to initiation. Scale bar is 100 μm. Slight reduction in size on heating is consistent with dehydration of the PNIPAM shell on the PEG microgels.

FIG. 36 is a plot showing Albumin production in HepC3A hepatocytes printed in heat sterilized LLS microgels.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims. Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definitions

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject-matter.

The term “about”, when used herein in reference to a value, refers to a value that is similar, in context to the referenced value. In general, those skilled in the art, familiar with the context, will appreciate the relevant degree of variance encompassed by “about” in that context. In an embodiment, “about” means a range encompassing +/−10% of the reference value. In an embodiment, “about” means a range encompassing +/−5% of the reference value.

Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

As used herein, the term “comparable” refers to two or more agents, entities, situations, sets of conditions, etc., that may not be identical to one another but that are sufficiently similar to permit comparison there between so that one skilled in the art will appreciate that conclusions can reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Those skilled in the art will appreciate that the term “composition”, as used herein, can be used to refer to a discrete physical entity that comprises one or more specified components. In general, unless otherwise specified, a composition can be of any form—e.g., gas, gel, liquid, solid, etc.

A composition or method described herein as “comprising” one or more named elements or steps is open-ended, meaning that the named elements or steps are essential to a particular aspect or embodiment, but other elements or steps can be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and can also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step can be substituted for that element or step.

As used herein, “Improved,” “increased” or “reduced”, or grammatically comparable comparative terms, indicate values that are relative to a baseline value or reference measurement. For example, in some embodiments, an assessed value achieved with an agent of interest may be “improved” relative to that obtained or expected in the absence of treatment or with a comparable reference agent or control. Alternatively, or additionally, in some embodiments, an assessed value achieved with an agent of interest may be “improved” relative to that obtained in the same subject or system under different conditions (e.g., prior to or after an event such as administration of an agent of interest), or in a different, comparable subject (e.g., in a comparable subject or system that differs from the subject or system of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

As used herein, “sample” refers to one or more biological substances (preferable a mammalian cell or plurality of mammalian cells or tissue [s]) whose position can be transported and/or physiologically maintained using systems and methods as described herein.

### DISCUSSION

Described herein are compositions, systems, and methods relating to three-dimensional (3D) bioprinted tissue models, in particular liver tissue models. In embodiments of the present disclosure, a plurality (two or more) of cell types can be mixed with a matrix material and deposited into a support medium thereby forming a planar cellular composition or tissue construct. In embodiments, the deposition can be with a 3D bioprinter. In embodiments, the deposition can be a planar shape having one dimension is between about 20 to 200 microns or two to ten cellular layers. In embodiments, the planar shape is a disc (circular or oval) shape with a width between about 20 to 200 microns or two to ten cellular layers. As described herein, planar cellular compositions can exhibit gene expression profiles, enzymatic function, and enzyme secretion similar to their in vivo counterparts, and can be kept healthy in tissue culture for 14 to 21 days or longer (or shorter, depending on the purpose).

Such tissue constructs can be embedded in support media of systems as described herein and perfused with growth media (statically or actively). Systems as described herein can further comprise bioreactors, which can be passive static bioreactors or perfusion-enabled bioreactors (that allow for active perfusion of cellular compositions with growth media via osmotic, negative, and/or positive pressure).

The growth media can contain known toxic or potential therapeutic compounds (such as small molecules, nucleic acids, proteins, viruses, and the like) that can induce cellular toxicity in the cells of compositions as described herein to create a model tissue injury, or alternatively repair cells following injury in models of disease (drug-induced liver injury, for example). After infusion of the growth media and perfusion of the cellular compositions, effluent media can be collected to analyze tissue secretions or cells themselves can be analyzed for changes as a result of infusion and perfusion.

### Planar Cellular Compositions (i.e., Tissue Constructs)

Described herein are 3D bioprinted tissue models (also referred to herein as planar cellular compositions or tissue constructs, or planar tissue constructs or discs or discoids, which are neither organoids nor spheroids).

In embodiments, tissue models as described herein comprise hepatocytes. In an embodiment, tissue models as described herein comprise endothelial cells. In an embodiment, tissue models as described herein comprise hepatocytes and endothelial cells in a ratio of 2:1 hepatocyte: endothelial cell. In embodiments, the hepatocytes, endothelial cells, or both are primary cells. In embodiments, the hepatocytes, endothelial cells, or both are human, rat, or mouse primary cells, although primary cells from other organisms may also be suitable. In additional embodiments, tissue models as described herein can further comprise non-parenchymal cells.

Tissue models as described herein can take on a number of geometric shapes (circular, oval, square, rectangular, etc.), so long as they are planar and have at least one physical dimension of about 20 to 200 microns or two to ten cellular layers. In embodiments, the tissue models are disc-shapes (circular or oval) having one physical dimension (a thickness) of about 200 microns. Such physical dimensions ensure that each cell can receive oxygen and avoid the tissue model becoming necrotic because of hypoxia-induced apoptotic or autophagic cellular death processes.

Tissue constructs as described herein can further comprise a matrix material. In embodiments, the matrix material comprises one or more collagens. In embodiments, the matrix material may be reconstituted liver matrix, Matrigel®, or a combination thereof.

In other aspects, tissue constructs as described herein are compositions that are a product of mixing two or more cell types together with a matrix material and bioprinting the mixture into a support medium, wherein the bioprinting is done in a spiral pattern starting from a center point and extending annularly and radially outward, leading to the formation of a 3D tissue construct that is disc-shaped. Should more than tissue model be bioprinted, the variation of diameter between the disc-shaped tissue models should be less than 5%.

### Cells

A variety of different cells can be using according to compositions, systems, and methods as described herein. As the skilled artisan would envisage, cells as described herein can be mammalian cells with an origin of any germ layer (mesodermal, endodermal, ectodermal, or placental).

In some embodiments, these can be normal human cells. In some embodiments, these can be primary cells. The cells that form tissue models as described herein may be mixture of cell types in suspension. Cells can be obtained from cell culture or biopsy. Cells can be of one or more types, either differentiated cells, such as endothelial cells or parenchymal cells, including nerve cells, or undifferentiated cells, such as stem cells or embryonic cells. In embodiments, cells can be hepatocytes. In embodiments, cells can be endothelial cells, for example human umbilical vein endothelial cells (HUVECs). In other aspects, cells may be non-parenchymal cells, for example Kupffer cells or stellate cells.

### Methods of Making Tissue Models

Further described herein are methods of making tissue models according to the present disclosure. Methods of making tissue models as described herein can comprise mixing a first cell type, a second cell type, and a matrix material in suspension; loading the suspension into a bioprinter; printing the suspension into a support medium. Methods of making tissue models as described herein can further comprise providing a printing template preceding the printing where the printing template defines the shape which the tissue model will assume. In embodiments, the printing template is created by the user. In embodiments, the printing template comprises instructions to print one physical dimension of about 20 to about 200 microns. In embodiments, the printing template can be a spiral pattern to create a disc with a thickness of 200 microns. Methods of making tissue models as described herein can further comprise printing the suspension in a bioreactor. Methods of making tissue models as described herein can further describe printing two or more suspension in two or more bioreactors, each suspension having a unique bioreactor, and discarding any printed suspension that varies in diameter more than 5% from two or more other printed suspensions.

### Systems

Systems as described herein can comprise a bioreactor and a support medium. Systems as described herein can further comprise a cellular growth medium (also described herein as a growth medium or media. Systems as described herein can further comprise a tissue model as described herein. Systems as described herein can further comprise a growth medium perfusion mechanism, for example a pump that provides positive or negative pressure.

Systems as described herein can further comprise a bioprinter as known in the art, for example, the CELLINK BIO X™ printer or the Advanced Solutions BioBot® series printers.

### Bioreactor[s]

Devices and systems as described herein can comprise one or more bioreactors. In embodiments, bioreactors as described herein are a well of a tissue culture plate (for example a 6-well, 24-well, 48-well, 96-well, or 384-well tissue culture plate such as those known in the art). In embodiments, bioreactors have a bottom surface that can be flat, tapered, concave, or convex. The skilled artisan would understand that a wide variety of bioreactors can be utilized according to the present disclosure.

In additional aspects, bioreactors as described herein can be perfusion-enabled bioreactors such as those known and described in the art, for example, positive-, negative-or osmotic-pressure driven perfusion enabled bioreactors. In other aspects, bioreactors as described herein can be conventional static bioreactors in which no growth media perfusion through the support media and/or cellular compositions as described herein is pressure driven.

### Support Medium

In certain aspects, systems as described herein can comprise support medium in which tissue constructs as described herein can be embedded.

In embodiments, and without intending to be limiting, the support medium can comprise agar, gelatin, Matrigel®, or other such semi-solid support mediums known in the art of tissue culture. Additional examples include agarose, gelatins or various types, solidified collagen, Liquid Like Solids (as described herein). Any support material capable of perfusion through its bulk under a pressure gradient could be used here. Chopped up seaweed with plankton interspersed would work if that was the desired sample.

In an embodiment, the support medium can be a liquid-like solid (LLS) three-dimensional (3D) cell growth medium, as further described below.

Liquid-like solid (LLS) three-dimensional (3D) cell growth medium for use in with the disclosed bio-manipulator system is disclosed in WO2016182969A1 by Sawyer et al., which is incorporated by reference in its entirety for the description of how to make and uses this LLS medium.

Briefly, the 3D cell growth medium may comprise hydrogel particles dispersed in a liquid cell growth medium. Any suitable liquid cell growth medium may be used; a particular liquid cell growth medium may be chosen depending on the types of cells which are to be placed within the 3D cell growth medium. For example, suitable cell growth medium may be human cell growth medium, murine cell growth medium, bovine cell growth medium or any other suitable cell growth medium. Depending on the particular embodiment, hydrogel particles and liquid cell growth medium may be combined in any suitable combination. For example, in some embodiments, a 3D cell growth medium comprises approximately 0.5% to 1% hydrogel particles by weight.

In accordance with some embodiments, the hydrogel particles may be made from a bio-compatible polymer. A wide variety of hydrogel particles may be employed as a support medium as described herein so long as they are inert, biocompatible, and exhibit good pore space (between 10 nm and 10 micrometers) when packed and/or swollen with culture medium (aka growth medium).

The hydrogel particles may swell with the liquid growth medium to form a granular gel material. Depending on the particular embodiment, the swollen hydrogel particles may have a characteristic size at the micron or submicron scales. For example, in some embodiments, the swollen hydrogel particles may have a size between about 0.1 μm and 100 μm. Furthermore, a 3D cell growth medium may have any suitable combination of mechanical properties, and in some embodiments, the mechanical properties may be tuned via the relative concentration of hydrogel particles and liquid cell growth medium. For example, a higher concentration of hydrogel particles may result in a 3D growth medium having a higher elastic modulus and/or a higher yield stress.

According to some embodiments, the 3D cell growth medium may be made from materials such that the granular gel material undergoes a temporary phase change due to an applied stress (e.g., a thixotropic or “yield stress” material). Such materials may be solids or in some other phase in which they retain their shape under applied stresses at levels below their yield stress. At applied stresses exceeding the yield stress, these materials may become fluids or in some other more malleable phase in which they may alter their shape. When the applied stress is removed, yield stress materials may become solid again. Stress may be applied to such materials in any suitable way. For example, energy may be added to such materials to create a phase change. The energy may be in any suitable form, including mechanical, electrical, radiant, or photonic, etc.

Regardless of how cells are placed in the medium, the yield stress of the yield stress material may be large enough to prevent yielding due to gravitational and/or diffusional forces exerted by the cells such that the position of the cells within the 3D growth medium may remain substantially constant over time. As described in more detail below, placement and/or retrieval of groups of cells may be done manually or automatically.

A yield stress material as described herein may have any suitable mechanical properties. For example, in some embodiments, a yield stress material may have an elastic modulus between approximately 1 Pa and 1000 Pa when in a solid phase or other phase in which the material retains its shape under applied stresses at levels below the yield stress. In some embodiments, the yield stress required to transform a yield stress material to a fluid-like phase may be between approximately 1 Pa and 1000 Pa. In some embodiments, the yield stress may be on the order of 10 Pa, such as 10 Pa +/−25%. When transformed to a fluid-like phase, a yield stress material may have a viscosity between approximately 1 Pa s and 10,000 Pas. However, it should be understood that other values for the elastic modulus, yield stress, and/or viscosity of a yield stress material are also possible, as the present disclosure is not so limited.

A group of cells may be placed in a 3D growth medium made from a yield stress material via any suitable method. For example, in some embodiments, cells may be injected or otherwise placed at a particular location within the 3D growth medium with a syringe, pipette, or other suitable placement or injection device. In some embodiments an array of automated cell dispensers may be used to inject multiple cell samples into a container of 3-D growth medium. Movement of the tip of a placement device through the 3D growth medium may impart a sufficient amount of energy into a region around the tip to cause yielding such that the placement tool may be easily moved to any location within the 3D growth medium. In some instances, a pressure applied by a placement tool to deposit a group of cells within the 3D growth medium may also be sufficient to cause yielding such that the 3D growth medium flows to accommodate the group of cells. Movement of a placement tool may be performed manually (e.g., “by hand”) or may performed by a machine or any other suitable mechanism.

In some embodiments, multiple independent groups of cells may be placed within a single volume of a 3D cell growth medium. For example, a volume of 3D cell growth medium may be large enough to accommodate at least 2, at least 5, at least 10, at least 20, at least 50, at least 100, at least 1000, or any other suitable number of independent groups of cells. Alternatively, a volume of 3D cell growth medium may only have one group of cells. Furthermore, it should be understood that a group of cells may comprise any suitable number of cells, and that the cells may of one or more different types.

Depending on the particular embodiment, groups of cells may be placed within a 3D cell growth medium according to any suitable shape, geometry, and/or pattern. For example, independent groups of cells may be deposited as spheroids, and the spheroids may be arranged on a 3D grid, or any other suitable 3D pattern. The independent spheroids may all comprise approximately the same number of cells and be approximately the same size, or alternatively different spheroids may have different numbers of cells and different sizes. In some embodiments, cells may be arranged in shapes such as embryoid or organoid bodies, tubes, cylinders, toroids, hierarchically branched vessel networks, high aspect ratio objects, thin closed shells, or other complex shapes which may correspond to geometries of tissues, vessels or other biological structures.

According to some embodiments, a 3D cell growth medium made from a yield stress material may enable 3D printing of cells to form a desired pattern in three dimensions. For example, a computer-controlled injector tip may trace out a spatial path within a 3D cell growth medium and inject cells at locations along the path to form a desired 3D pattern or shape. Movement of the injector tip through the 3D cell growth medium may impart sufficient mechanical energy to cause yielding in a region around the injector tip to allow the injector tip to easily move through the 3D cell growth medium, and also to accommodate injection of cells. After injection, the 3D cell growth medium may transform back into a solid-like phase to support the printed cells and maintain the printed geometry. However, it should be understood that 3D printing techniques are not required to use a 3D growth medium as described herein.

According to some embodiments, a 3D cell growth medium may be prepared by dispersing hydrogel particles in a liquid cell growth medium. The hydrogel particles may be mixed with the liquid cell growth medium using a centrifugal mixer, a shaker, or any other suitable mixing device. During mixing, the hydrogel particles may swell with the liquid cell growth medium to form a material which is substantially solid when an applied shear stress is below a yield stress, as discussed above. After mixing, entrained air or gas bubbles introduced during the mixing process may be removed via centrifugation, agitation, or any other suitable method to remove bubbles from 3D cell growth medium.

In some embodiments, preparation of a 3D cell growth medium may also involve buffering to adjust the pH of a hydrogel particle and liquid cell growth medium mixture to a desired value. For example, some hydrogel particles may be made from polymers having a predominantly negative charge which may cause a cell growth medium to be overly acidic (have a pH which is below a desired value). The pH of the cell growth medium may be adjusted by adding a strong base to neutralize the acid and raise the pH to reach the desired value. Alternatively, a mixture may have a pH that is higher than a desired value; the pH of such a mixture may be lowered by adding a strong acid. According to some embodiments, the desired pH value may be in the range of about 7.0 to 7.4, or, in some embodiments 7.2 to 7.6, or any other suitable pH value which may, or may not, correspond to in vivo conditions. The pH value, for example may be approximately 7.4. In some embodiments, the pH may be adjusted once the dissolved CO₂ levels are adjusted to a desired value, such as approximately 5%.

Yield stress can be measured by performing a strain rate sweep in which the stress is measured at many constant strain rates. Yield stress can be determined by fitting these data to a classic Herschel-Bulkley model (σ=σ_{y}+kγⁿ). (b) To determine the elastic and viscous moduli of non-yielded LLS media, frequency sweeps at 1% strain can be performed. The elastic and viscous moduli remain flat and separated over a wide range of frequency, behaving like a Kelvin-Voigt linear solid with damping. Together, these rheological properties demonstrate that a smooth transition between solid and liquid phases occurs with granular microgels, facilitating their use as a 3D support matrix for cell printing, culturing, and assaying.

An example of a hydrogel with which some embodiments may operate is a carbomer polymer, such as Carbopol®. Carbomer polymers may be polyelectrolytic and may comprise deformable microgel particles. Carbomer polymers are particulate, high-molecular-weight crosslinked polymers of acrylic acid with molecular weights of up to 3-4 billion Daltons. Carbomer polymers may also comprise co-polymers of acrylic acid and other aqueous monomers and polymers such as poly-ethylene-glycol.

While acrylic acid is a common primary monomer used to form polyacrylic acid the term is not limited thereto but includes generally all α-β unsaturated monomers with carboxylic pendant groups or anhydrides of dicarboxylic acids and processing aids as described in U.S. Pat. No. 5,349,030. Other useful carboxyl containing polymers are described in U.S. Pat. No. 3,940,351, directed to polymers of unsaturated carboxylic acid and at least one alkyl acrylic or methacrylic ester where the alkyl group contains 10 to 30 carbon atoms, and U.S. Pat. Nos. 5,034,486; 5,034,487; and 5,034,488; which are directed to maleic anhydride copolymers with vinyl ethers. Other types of such copolymers are described in U.S. Pat. No. 4,062,817 wherein the polymers described in U.S. Pat. No. 3,940,351 contain additionally another alkyl acrylic or methacrylic ester and the alkyl groups contain 1 to 8 carbon atoms. Carboxylic polymers and copolymers such as those of acrylic acid and methacrylic acid also may be cross-linked with polyfunctional materials as divinyl benzene, unsaturated diesters and the like, as is disclosed in U.S. Pat. Nos. 2,340,110; 2, 340, 111; and 2,533,635. The disclosures of all of these U.S. Patents are hereby incorporated herein by reference for their discussion of carboxylic polymers and copolymers that, when used in polyacrylic acids, form yield stress materials as otherwise disclosed herein. Specific types of cross-linked polyacrylic acids include carbomer homopolymer, carbomer copolymer and carbomer interpolymer monographs in the U.S. Pharmocopia 23 NR 18, and Carbomer and C10-30 alkylacrylate crosspolymer, acrylates crosspolymers as described in PCPC International Cosmetic Ingredient Dictionary & Handbook, 12th Edition (2008).

Carbomer polymer dispersions are acidic with a pH of approximately 3. When neutralized to a pH of 6-10, the particles swell dramatically. The addition of salts to swelled Carbomer can reduce the particle size and strongly influence their rheological properties. Swelled Carbomers are nearly refractive index matched to solvents like water and ethanol, making them optically clear. The original synthetic powdered Carbomer was trademarked as Carbopol® and commercialized in 1958 by BF Goodrich (now known as Lubrizol), though Carbomers are commercially available in a multitude of different formulations.

Hydrogels may include packed microgels-microscopic gel particles, ˜5 μm in diameter, made from crosslinked polymer. The yield stress of Carbopol® is controlled by water content. Carbopol® yield stress can be varied between roughly 1-1000 Pa. Thus, both materials can be tuned to span the stress levels that cells typically generate. As discussed above, while materials may have yield stresses in a range of 1-1000 Pa, in some embodiments it may be advantageous to use yield stress materials having yield stresses in a range of 1-100 Pa or 10-100 Pa. In addition, some such materials may have thixotropic times less than 2.5, less than 1.5 seconds, less than 1 second, or less than 0.5 seconds, and greater than 0.25 seconds or greater than 0.1 seconds, and/or thixotropic indexes less than 7, less than 6.5, or less than 5, and greater than 4, or greater than 2, or greater than 1.

Yield stresses of less than 100 pascals are advantageous as they prevent the formation of unwanted crevasses in the 3D culture medium that detrimentally affects flow of fluid (and nutrient delivery/retrieval) throughout the material. Additionally, yield stresses in this range have advantages for the culture of cells, such as efficient waste retrieval and the ability of cells to expand in their environment without being unnecessarily constrained.

In certain aspects, hydrogel particles are spherical. In certain aspects, hydrogel particles as described herein can be anionic, cationic, zwitterionic, or charge-neutral. In certain aspects, a charge can be a charge on the surface of a hydrogel particle or sphere. In embodiments, the hydrogel particles comprise poly-ethylene glycol (PEG).

In embodiments, the hydrogel particles can be functionalized, i.e., chemically modified to provide for a given chemical reactivity according to the modification that is different than the native unfunctionalized reactivity. In embodiments, hydrogel particles as described herein can be thiol-functionalized (with a thiol R-S-H group or thiol-ene group), coated with gelatin, functionalized with RGD peptide, functionalized with poly-lysine, or any combination therein.

### Growth Medium

Growth medium or media (for example liquid medium) compositions as known in the art, that can be employed in addition to the support medium (also referred to herein as 3D growth medium) as described herein, must be considered from two perspectives relating to the desired tissue and/or cells that are to be utilized in devices and systems as described herein: basic nutrients (sugars, amino acids, and the like) and growth factors/cytokines. Examples of common growth medium compositions include those based on media such as Dulbecco's Modified Eagle Medium (DMEM) that can be supplemented with other components, such as non-essential amino acids, antibiotics or antibiotic cocktails (for example penicillin-streptomycin), and nutrients (such as those stemming from fetal bovine serum or other sources). The skilled artisan would understand that the specific growth medium to be employed in systems and methods as described herein will be dependent on the biological samples utilized and could tailor the growth medium accordingly based on their level of ordinary skill and knowledge in the art.

### Applications

Without intending to be limiting, systems and methods as described herein have many different applications, such as toxicity studies; metabolic studies of drug metabolism; assisting with the identification of markers of disease; assessing efficacy of anti-cancer therapeutics; testing gene therapy vectors; drug development; screening; screening in tissue models of disease; studies on biotransformation, clearance, metabolism, and activation of xenobiotics; studies on bioavailability and transport of chemical agents across epithelial layers; studies on bioavailability and transport of biological agents across epithelial layers; studies on acute basal toxicity of chemical agents; studies on acute local or acute organ-specific toxicity of chemical agents; studies on chronic basal toxicity of chemical agents; studies on chronic local or chronic organ-specific toxicity of chemical agents; studies on teratinogenicity of chemical agents; studies on genotoxicity, carcinogenicity, and mutagenicity of chemical agents; detection of infectious biological agents and biological weapons; detection of harmful chemical agents and chemical weapons; studies on infectious diseases; studies on the efficacy of chemical agents to treat disease; studies on the efficacy of biological agents to treat disease; studies on the optimal dose range of agents to treat disease; prediction of the response of organs in vivo to biological agents; prediction of the pharmacokinetics of chemical or biological agents; prediction of the pharmacodynamics of chemical or biological agents; studies concerning the impact of genetic content on response to agents; studies on gene transcription in response to chemical or biological agents; studies on protein expression in response to chemical or biological agents; studies on changes in metabolism in response to chemical or biological agents.

### Specific Embodiments

Embodiment 1. A planar cellular composition, comprising hepatocytes and endothelial cells in a matrix material, wherein the hepatocytes and endothelial cells are present in a ratio of about 2:1 to 1:1 hepatocytes:endothelial cells.
Embodiment 2. The planar cellular composition of embodiment 1, wherein the hepatocytes are primary cells.
Embodiment 3. The planar cellular composition of embodiment 1 or 2, wherein the endothelial cells are vascular endothelial cells.
Embodiment 4. The planar cellular composition of any one of embodiments 1 to 3, wherein the hepatocytes and endothelial cells are present in a ratio of about 2:1 hepatocytes:endothelial cells.
Embodiment 5. The planar cellular composition of any one of embodiments 1 to 4, wherein the matrix material comprises collagen.
Embodiment 6. The planar cellular composition of any one of embodiments 1 to 5, wherein the planar cellular composition has at least one physical dimension between about 5 to 20 cells in thickness.
Embodiment 7. The planar cellular composition of any one of embodiments 1 to 5, wherein the planar composition has a circular or oval shape.
Embodiment 8. The planar cellular composition of embodiment 6, wherein the circular or oval shape has a about 0.5 to 10 mm diameter, including about 1.5 to 2.5 mm in diameter.
Embodiment 9. The planar cellular composition of any one of embodiments 1 to 8, wherein the endothelial cells form one or more sinusoidal structures that are not organized into a network.
Embodiment 10. A method of creating a planar cellular composition, comprising:
   mixing hepatocytes, endothelial cells, and a matrix material in a cellular growth medium to form a cellular mixture, wherein the hepatocytes and endothelial cells are present in a ratio of about 15:10 to 20:15 hepatocytes:endothelial cells;
   placing the cellular mixture in a support medium to form a planar cellular composition about 5 to 20 cells in thickness and about 0.5 to 10 mm diameter; and
   culturing the cellular mixture under physiological growth conditions to produce the planar cellular composition.
Embodiment 11. The method of embodiment 9, wherein the hepatocytes are primary cells.
Embodiment 12. The method of embodiment 9 or 11, wherein the endothelial cells are vascular endothelial cells.
Embodiment 13. The method of any one of embodiments 9 to 12, wherein the hepatocytes and endothelial cells are present in a ratio of about 2:1 hepatocytes:endothelial cells.
Embodiment 14. The method of any one of embodiments 9 to 13, wherein the planar composition has a circular or oval shape.
Embodiment 15. The method cellular composition of embodiment 14, wherein the circular or oval shape has a about 1.5 to 2.5 mm in diameter.
Embodiment 16. The method of any one of embodiments 9 to 15, wherein the matrix material comprises collagen.
Embodiment 17. The method of any one of embodiments 9 to 16, wherein the support material comprises polymeric packed microgel particles.
Embodiment 18. The method of any one of embodiments 9 to 16, wherein the support material comprises poly-ethylene glycol (PEG) microgels.
Embodiment 19. The method of any one of embodiments 9 to 18, wherein the cellular growth medium comprises hepatocyte growth factor (HGF) but does not comprise vascular endothelial growth factor (VEGF).
Embodiment 20. The method of any one of embodiments 9 to 19, wherein the placing is performed with a three-dimensional (3D) printer.
Embodiment 21. The method of any one of embodiments 9 to 20, wherein the placing is in a spiral pattern extending radially and annularly around an initial placement point.
Embodiment 22. A planar cellular composition produced according to the method of any one of embodiments 9 to 21.
Embodiment 23. A tissue culture system, comprising:
   a planar cellular composition of any one of embodiments 1 to 9 embedded in a support medium; and
   a bioreactor.
Embodiment 24. The system of embodiment 23, further comprising a cellular growth medium.
Embodiment 25. The system of embodiment 24, wherein the cellular growth medium comprises hepatocyte growth factor (HGF) but does not comprise vascular endothelial growth factor (VEGF).
Embodiment 26. The system of any one of embodiments 23 to 25, wherein the bioreactor is a perfusion-enabled bioreactor.
Embodiment 27. The system of any one of embodiments 23 to 26, wherein support medium comprises polymeric packed microgel particles.
Embodiment 28. The system of any one of embodiments 23 to 26, wherein the support medium comprises poly-ethylene glycol (PEG) microparticles.
Embodiment 29. A method for screening candidate agents, comprising contacting the candidate agent to the planar cellular composition of any one of embodiments 1 to 9 and evaluating one or more properties of the planar cellular com position.
Embodiment 30. The method of embodiment 29, wherein the candidate agent is a therapeutic candidate selected from the group consisting of a small molecule, a protein, and a nucleic acid.
Embodiment 31. The method of embodiment 30, wherein the candidate agent is a toxicity candidate selected from the group consisting of a small molecule, a protein, a nucleic acid, and a pathogen.
Embodiment 32. The method of any one of embodiments 29 to 31, wherein the one or more properties comprises albumin synthesis, urea production, or a combination thereof.

A number of embodiments of the present disclosure have been described. Nevertheless, it will be understood that various modifications may be made without departing from the spirit and scope of the present disclosure. Accordingly, other embodiments are within the scope of the following claims.

In addition, the following embodiments and features can be incorporated into one or more aspects or embodiments as provided herein. The following are provided to illustrate additional features that can be incorporated together with embodiments provided above and herein as well as with one or more of each other. The present disclosure is not limited to each feature independently, rather various combinations of one or more of these features with one or more of the features disclosed above and herein in contemplated.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

### Project Summary

Animal models and in vitro cell culture approaches are widely used to test compounds for potential liver toxicity during the drug development process. However, numerous limitations of both general approaches are widely recognized; animal models often fail to predict toxicity in humans while in vitro models lack the complexity of real tissue that is required for proper function and the related response to compounds. Thus, to increase the pace and lower the costs of drug development, there is a critical need for technologies capable of predicting the toxicity of compounds. To address this critical need, in this project an in vitro liver tissue model was developed for use in toxicological investigation. To manufacture functional liver tissue models, 3D bioprinting was combined with new 3D culture materials to create reproducible cellular structures that remain viable over the course of at least 21 days. With such capabilities, assays for characterizing 3D printed tissue function were developed and tested. These assays included the measurement of total cell numbers over time, albumin and urea synthesis rate, gene expression profiles, and function for key enzymes responsible for the metabolism of the majority of pharmaceutical drugs. With these metrics tissue compositions and culture conditions that produced functional tissues could be identified. Overall, the best tissues performed as proposed, stably secreting requisite levels of albumin and urea over a 7-10 day window, expressing 56 ADME genes at levels commensurate with human liver, and demonstrating metabolic enzyme function.

### INTRODUCTION

Current animal models used in toxicology can only predict a fraction of drugs that may induce liver injury in humans, leading to costly clinical development failures, black box warnings, or withdrawal of drugs from the market. Thus, in vitro human systems are being developed to better predict drug-induced liver injury (DILI) earlier in drug development. However, many major challenges remain for human in vitro systems. For example, monolayer cell culture's utility in predicting liver-specific responses is limited, yet it is broadly used in pharmaceutical/biopharmaceutical discovery and development. While more advanced approaches can have functional characteristics (e.g., albumin and urea production, drug metabolism capabilities) closer to those observed in vivo, these are generated using imprecise tools such as hanging drops, which leads to high variability, prohibiting Critical Quality Attribute (CQA) quantification of final products. Therefore, industry needs reliable 3D microtissue culture systems that are a) relatively high throughput. B) stable for ≥14 to 28 days, c) reproducible and transferable, d) able to be assessed by meaningful endpoints, e) cost effective, f) good mimics of in vivo systems, g) capable of good human dose/exposure prediction, and h) able to differentiate good compounds from bad (efficacy and/or safety), avoiding the false implication of good drugs. This type of 3D microtissue culture system would integrate with methods and tools for achieving structured cell assemblies, such as bioprinting technologies. Generating a manufacturing process for a 96-well plate-based microtissue screening platform will enable the rapid development of cell culture, biofabrication, metrology and transport/storage solutions that minimize product variability and facilitate product quality assessment for centralized manufacturing that will later be applicable to patient-derived tissues for therapeutic applications.

Numerous hepatic model systems and culture approaches have been developed using advanced methods. In 2D, monolayers of iPSC-derived human hepatocytes and bioengineered micropatterned co-cultures of primary hepatocytes and fibroblasts have exhibited some morphological and functional features of in vivo liver tissue [2,3]. To simulate physiologically relevant fluid shearing and solute transport, dynamic flow has been applied to hepatocyte layers sandwiched between collagen gels [4]. Recognizing the limitations of 2D culture methods, research has been performed on 3D spheroid culture of primary hepatocytes [5], 3D bioprinted liver tissue in transwells [6], and 3D printed scaffolds seeded with hepatocytes. The most significant limitations of these 3D methods is their inability to be scaled up to produce large numbers of 3D microtissues with high degrees of reproducibility in size, shape, composition, or controlled distribution of different cell types or ECM components. The 3D printing technology developed in this project overcomes these limitations, enabling the manufacture of microtissue at the scale required by industry, and accelerating the path to creating liver microtissues predictive of DILI in vivo.

To develop functional liver tissue models, four major milestone tasks were pursued that focused on (1) biofabrication, (2) perfusion, (3) tissue composition and cell selection, and (4) functional characterization. Within milestone task #1, a multi-material printer was modified (subtask 1.1) and tested (subtask 1.2). Within milestone task #2, a new microgel-based 3D printing and culture medium was developed (subtask 2.1), and new assays were developed for testing liver microtissues cultured within this environment (subtask 2.2). These new assays were used to monitor liver microtissue function (subtask 2.3) and the sensitivity of tissue function to perfusion conditions were studied (subtask 2.4). Within milestone task #3, different combinations of hepatocytes, endothelial cells, and cholangiocytes were tested in different media and extracellular matrix (ECM) microenvironments (subtask 3.1). Multilayer structures were fabricated (subtask 3.2) and different combinations of cells ECM were tested for their capacity to induce the assembly of a sinusoid-like structure (subtask 3.3). Within milestone task #4, an ADME gene panel of 56 CYP450 phase 1, phase 2, and drug transporter genes was performed (subtask 4.1) and metabolic enzymatic function related to a subset of these genes was tested (subtask 4.2).

### Methods

Additional aspects of methods as described herein and data relating to subtasks referenced above can be found in the Examples below.

### Results

One set of performance metrics was focused on the quality of the biofabrication technique: (1) the standard deviation of the longest linear dimension of tissues was to be less than 5% of the mean value; (2) the mean error of the longest dimension of tissues was to be less than 50 micrometers per 1 mm of linear length; (3) relative error tolerance on needle positioning was to be less than 50 microns per 1 mm of linear length. These performance metrics were met as previously reported (report 3). Another performance metric focused on the use of iPSC derived hepatocytes in 3D printed tissues. It was consistently found that iPSC derived hepatocytes did not meet the performance targets (report 4), so primary human hepatocytes were used for the final stages of the project. The final stages of the project were performed during the extension period of the project. Data collected during this period continued to be analyzed after March 31, so here it is reported the final outcomes of the 56 ADME gene panel, the production rates and stability of Albumin and Urea, and assessments of the metabolic enzymatic function.

### Albumin and Urea Production Rates & Stability

After settling on the conditions to be used for the 3D printed static culture human hepatocytes: HUVEC co-culture model, two experiments were performed (one in February 2021 and another in March 2021) to assess albumin and urea production rates and reproducibility of these parameters. Albumin and Urea levels were measured from the supernatants at various time points and the rate of production calculated from these values.

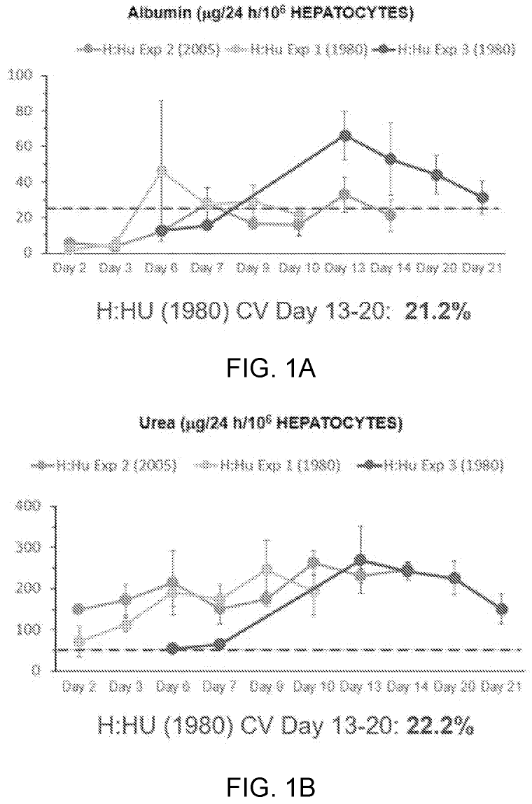

In the February experiment (FIGS. 1A-1B), results have shown that albumin and urea synthesis rates by the human hepatocyte: Huvec cells co-cultures using hepatocytes sourced from Donor #1980 were above the necessary Stage 1 criteria (>25 μg/1million hepatocytes/24 hrs for albumin and >50 μg/1million hepatocytes/24 hrs for urea). In this experiment, supernatant samples were collected on D6, 7, 13, 14, 20 and 21 for albumin and urea analyses. The intraexperiment C.V. for both albumin and urea levels met the criteria of <30% for the samples taken on D13, 14, and 20 (C.V.=21.2% and 22.2%, respectively). The performance from Donor #1980 was higher than Donor #2005, and so was selected for further downstream experiments.

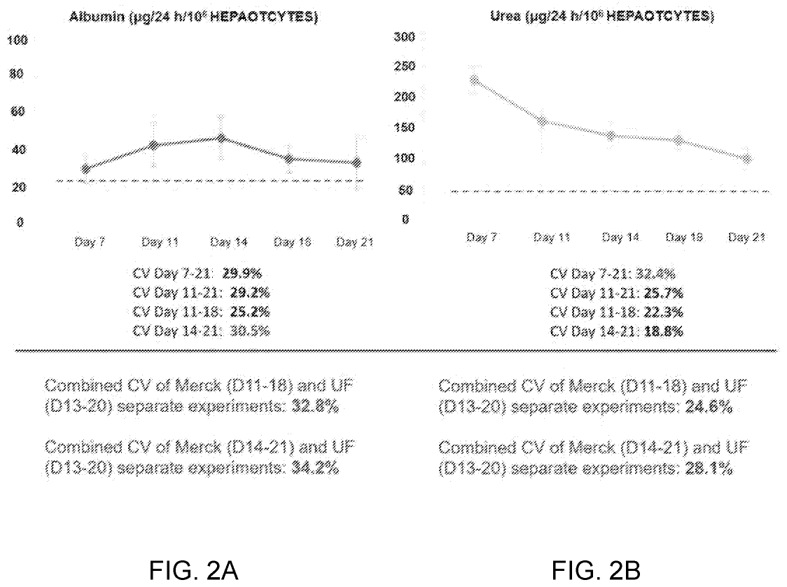

In the March experiment (FIGS. 2A-2B), the co-culture model using hepatocytes sourced from Donor #1980 showed that albumin exceeded the threshold criteria and multiple stable timeframes (e.g. C.V. <30% intra-experiment variability within a run across at least a 7 day timeframe) were identified within D7-21, D11-21, and D11-18, but not in the D14-21 range. Similarly, the levels of urea also exceeded the threshold criteria and the required 7-day stable timeframe was identified for the D11-21, D11-18, and D14-21 ranges, but not for the D7-21 range. Based on this, the D11-18 range seems to provide the best balance between meeting the production rate criteria for both albumin and urea, while also maintaining those levels relatively stable.

When comparing the stability across independent experiments, the stage 1 target of C.V. <30% for Albumin (e.g., D11-20 range at 32.8%) was slight short, but it was able to meet the target for Urea (e.g., D11-20 range at 24.6%). Taken together, it appears that the optimal stability of the system is in the D11-18 timeframe.

### Phase I and Phase II Metabolic Enzymatic Function Assessment

To assess metabolic enzymatic function of the 3D printed Hepatocyte: HUVEC co-cultures, a probe substrate cocktail was used to monitor metabolite formation related to key enzymes involved in small molecule metabolism. The assessment focused on the following enzymes: CYP1A2, CYP2B6, CYP2C9, CYP2D6, CYP3A4 and UGT1A1, which are the ones responsible for metabolizing the majority of drugs. The rate of metabolite formation was used to elucidate functional enzyme activity in the 3D liver system.

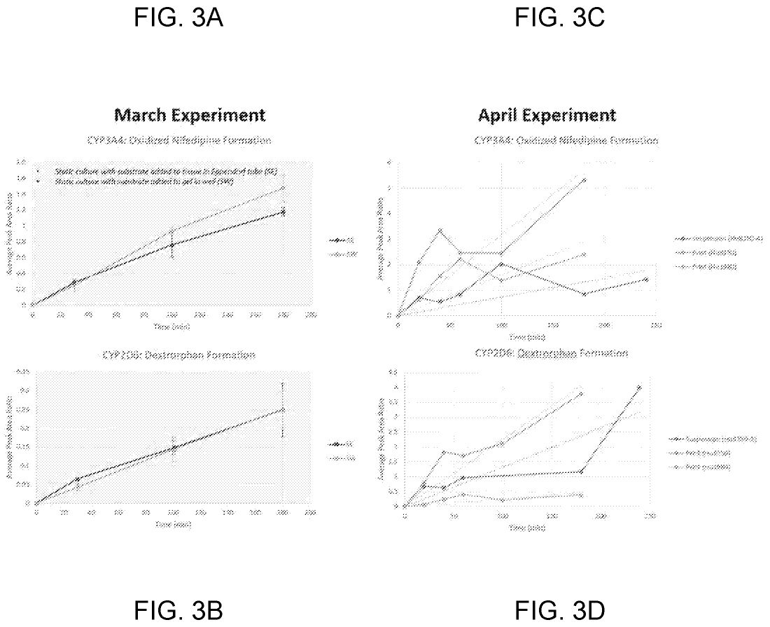

A total of three groups were initially assessed to investigate the mode of substrate incubation, and effect of perfusion in different possible assay conditions: 1) static culturing with substrate cocktail incubation in Eppendorf tubes, 2) static cultures with substrate incubation in the well with suspension gel, and 3) perfused cultures, with substrate incubation in Eppendorf tubes. Both static culture sample incubations (Conditions 1 and 2) provided measurable metabolites during the time course and were operating under linear kinetics. The incubation time for the perfusion samples (Condition 3) was insufficient to capture any metabolic activity perhaps due to insufficient substrate diffusion into the hepatocytes. Comparing the static incubations for CYP2C9, the in-well substrate incubation with suspension gel appeared to demonstrate a higher rate of metabolism (Condition 1 and 2 plotted in the March Experiment series in FIGS. 3A-3L). All other enzymes showed comparable rates across the time course, with slightly higher formation rates following in-well substrate incubation in most cases. Based on these results, a follow-up study was conducted with static cultures following in-well substrate incubation with suspension gel for an extended time course.

The goal of the follow-up assessment (April Experiment series in FIGS. 3A-3L) was to qualitatively compare enzymatic function between the static culture 3D microtissue (harvested at Day 15, post-print), using Donor #HU-8350, to freshly-thawed hepatocyte suspension controls (Also Donor #HU-8350), as the last of the remaining donor #1980, harvested in sub-optimal condition at Day 20 post-print. Both cultures and assays were performed using matched hepatocyte concentrations (˜20,000 cells/sample). Due to low sampling volumes, significant analytical noise and data variability was observed across all samples. While a direct comparison between incubation types was inconclusive, metabolic formation was observed for both 3D printed microtissues using both hepatocyte lots for all enzymes.

Gene Expression Analysis of 56 Absorption, Distribution, Metabolism, and Excretion (ADME)-related Genes

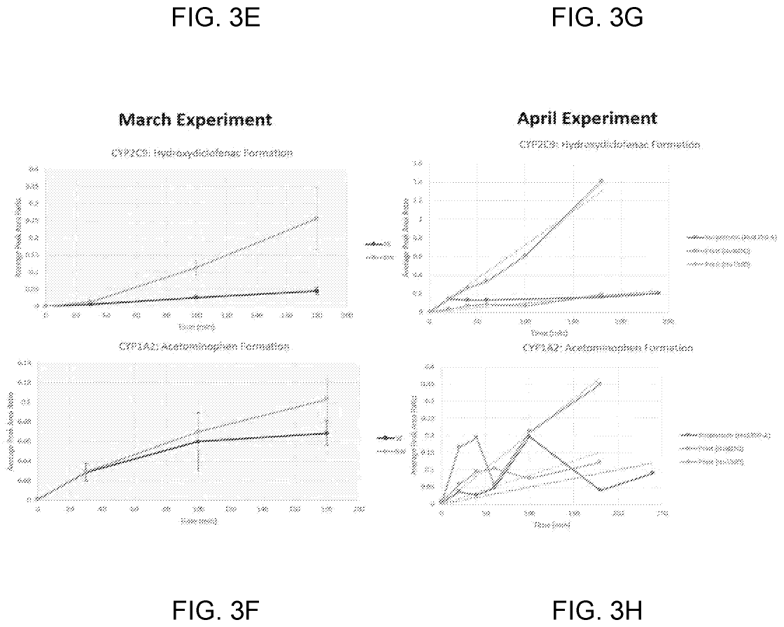

A total of 2 independent time course experiments were run to evaluate a panel of 56 ADME genes in the 3D printed models under static and perfusion conditions. The Stage 1 criteria was to determine if the genes could maintain stable expression for a period of 7 days at levels comparable to the Human HepatoPac® system and liver tissue samples from Human donors. Theoretical copy # for each gene was calculated based on the RT qPCR open array platform and described in FIGS. 4A-4K3. There was no clear benefit from the perfusion of the model compared to the static (no perfusion) condition using the current system. For all 56 ADME genes measured, the static model remained stable between the D14 and D21 timepoints for when RNA analysis was performed. Moreover, expression levels in this time frame were comparable to HepatoPac and human liver samples. The data from this second independent experiment replicate shown below is consistent with the first replicate data previously submitted in Milestone Report 4.1, and clearly achieves the Stage 1 criteria that has been set.

### DISCUSSION

Both the albumin and urea production rates and stability criteria in 2 independent experiments have been met. However, while the inter-experiment stability criteria (comparing Feb and Mar independent experiments) was met for urea synthesis, the criteria was not met for albumin production (32.8% CV, <30% required). The 56 ADME gene expression criteria has also been met in both of the 2 independent experiments and the metabolic enzymatic function data is encouraging, as it indicates that the 3D printed model activity is qualitatively comparable to that of primary hepatocytes. Nonetheless, the ADME function data was more variable than preferred (attributed to a sample processing change in the 2nd experiment) and a future follow-up experiment under optimized conditions will be needed to ensure that accurate quantitative conclusions on performance can be made.

### Example 2

### Overview and Principle Conclusions

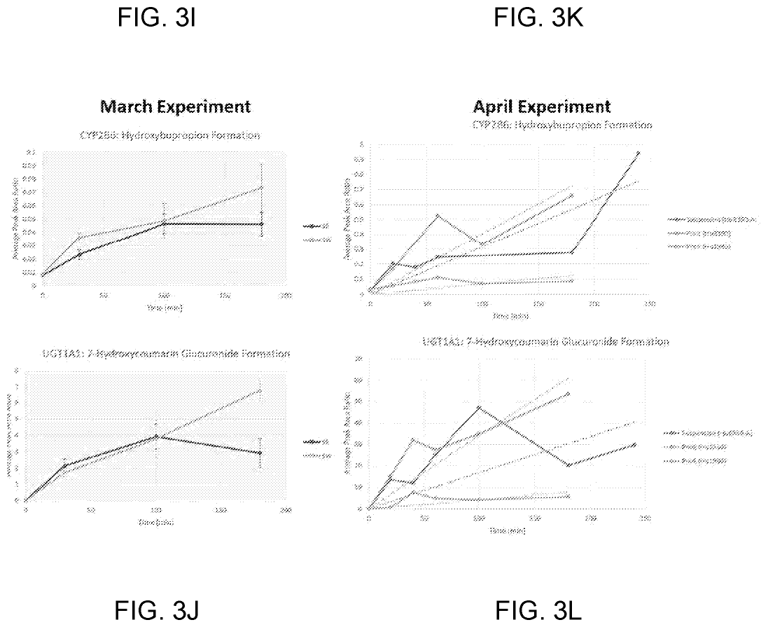

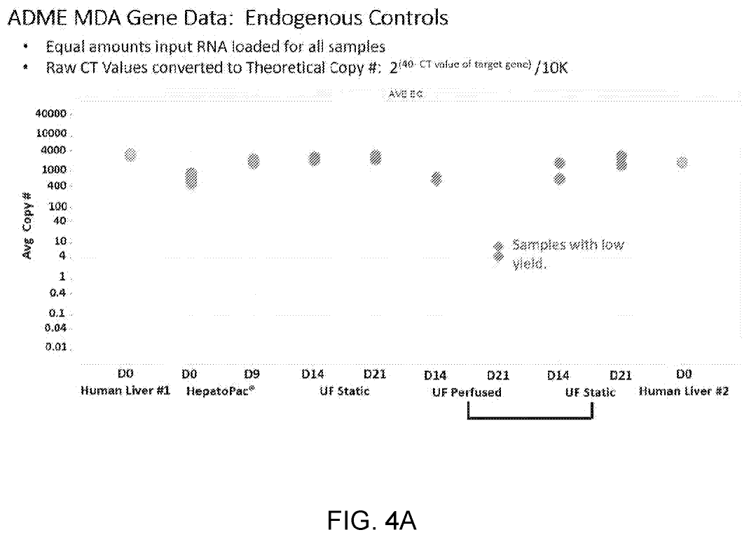

Seven different types of microgels having seven different chemical formulations were synthesized and tested for cytotoxicity (overview in FIG. 5). Three of these seven types of microgels were made by a precipitation polymerization method and the remaining four were made by an inverse emulsion polymerization method. The focus was on the potential impact different charged species may have on cell viability, and therefore synthesized cationic, anionic, zwitterionic, and uncharged microgels. Each of the charged types of microgels were synthesized using both polymerization routes; the neutral microgels were only prepared using the inverse emulsion polymerization route. Suitable candidate microgels to pursue in this project are the charge-neutral microgels (FIG. 6). The zwitterionic microgels performed nearly as well as PEG in terms of cell viability, but zwitterionic microgels exhibited swelling behavior sensitive to the ionic strength of their solvent. Since future work involves exchanging media containing a variety of different compounds, a microgel that is both insensitive to solvent composition and causes insignificant levels of cytotoxicity may represent the most robust material for future work. Thus, it was concluded that charge-neutral PEG microgels are a suitable candidate system for 3D printing, culturing, and assaying liver microtissues.

### Results

To test the impact charged groups may have on hepatocyte viability, cationic, anionic, and zwitterionic microgels were synthesized following the precipitation polymerization protocols previously established (see FIG. 5) [C. S. O'Bryan, et. al., ACS Applied Bio Materials, 2 1509 (2019)]. These microgels were made from the neutral polyacrylamide (pAAm) co-monomer, neutral polyethylene glycol diacrylate (PEGda) crosslinker, and additional charged co-monomers. Anionic microgels contained methacrylic acid (MAA); cationic microgels contained quaternized 2-(dimethylamino)ethyl methacrylate (qDMAEMA); zwitterionic microgels contained carboxybetaine methacrylate (CBMA). A schematic of the polymerization scheme is shown in FIG. 5 along with micrographs of the gel particles.

After comparing candidate chemistries for un-charged microgels, formulations entirely made from PEG polymers were pursued. PEG's excellent resistance to fouling and molecular adhesion, as well as its biocompatibility, motivated this choice. The solubility of PEG in organic solvents like ethanol and methanol led to a different polymerization route; there was a shift to an inverse emulsion polymerization in which PEG precursors were dispersed within aqueous droplets suspended in an organic liquid phase. Charge neutral microgels were synthesized from polyethylene glycol (PEG) (FIG. 6). By testing different formulations and synthesis protocols, highly spherical microgels were produced with a particle-size distribution peaked at around 10 μm diameter. See Appendix 1 for a detailed synthesis protocol.

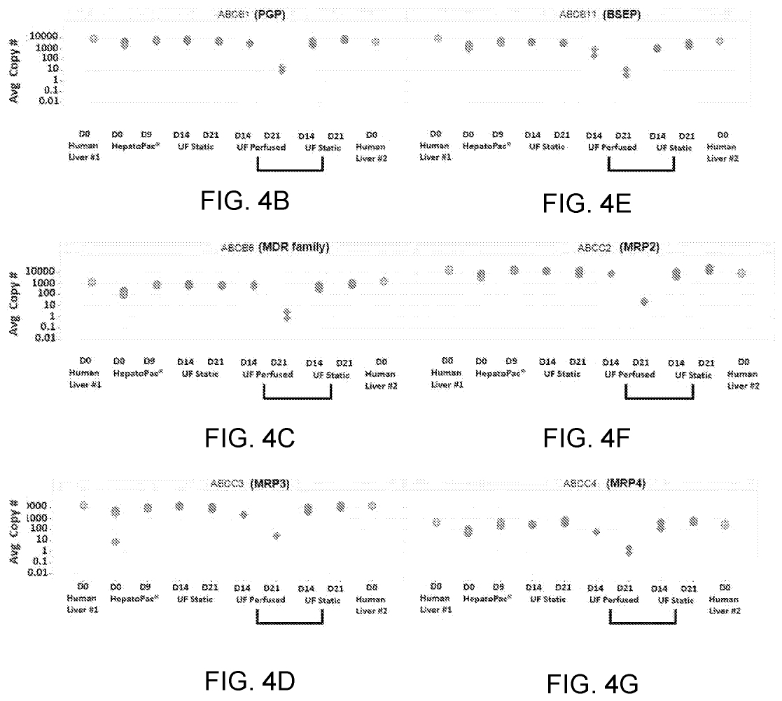

Since charged and uncharged microgels were synthesized using different methods that produced microgels with different shapes, an additional set of charged gels were synthesized using the inverse emulsion protocol. The same charged co-monomers previously used were incorporated into the new PEG microgel formulation. The chemical structures of the charged monomers and fully reacted copolymers are shown in FIG. 7 and example compositions are given in Appendix 2. Since the cationic qDMAEMA co-monomer requires an additional synthesis step, these details are also include in Appendix 2.

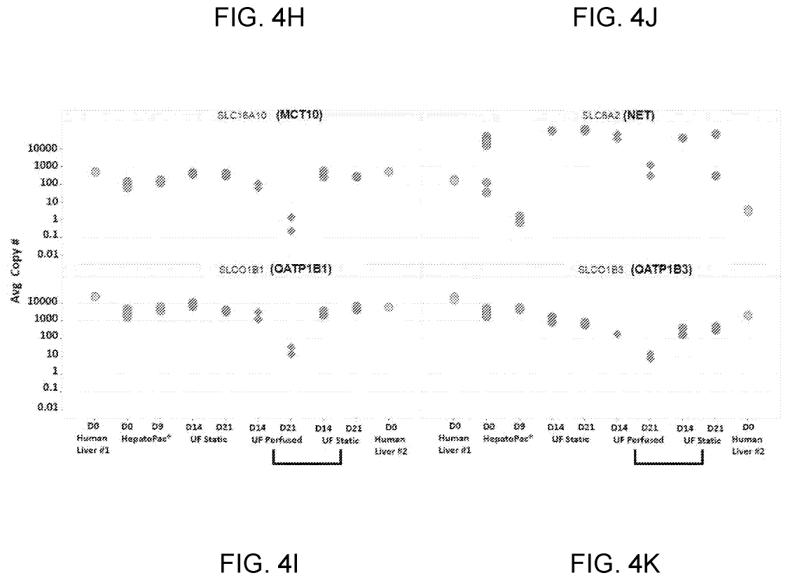

To compare these seven different microgel formulations in terms of their suitability for culture of 3D bio-printed liver structures, short-term (0-24 h) cytotoxicity assays were performed and the results compared to control samples in pure liquid media (no microgels). Cytotoxicity was measured using a standard live-dead assay. Cells were plated on a standard culture surface and left to attach and spread for approximately 24 hours. The liquid media was then replaced with microgels swollen in liquid media. The percent live cells were measured between one and three hours after the media exchange (labeled 0 h in FIG. 8) and also after 24 hours. For each of the seven microgel formulations, cell viability was measured on N=3 different samples at three different fields of view within each sample. The mean and standard deviation across measurements for each microgel formulation are shown in FIG. 8. Anionic and cationic microgels from the precipitation polymerization group were very cytotoxic to HepaRG hepatocytes. By contrast, the same chemical formulations made from inverse emulsion polymerization were far less cytotoxic and statistically equivalent to the liquid media control group. For both cases, the zwitterionic gels were a suitable charged species, performing nearly as well as the control samples. The uncharged PEG microgels always performed better than charged microgels (see FIG. 8). It was hypothesize that difference in cytotoxicity between microgels prepared by precipitation polymerization and those prepared by inverse emulsion polymerization arises from microgel shape. Microgels from inverse emulsions are perfectly spherical, while microgels from precipitation polymerization are irregularly shaped. The perfectly spherical microgels pack onto the cell surface like a layer of hard spheres, making far fewer direct contact points than the irregularly shaped gels, that more uniformly blanket the cells.

### Outcome

The uncharged PEG gels prepared by inverse emulsion polymerization exhibit the lowest levels of cytotoxicity, statistically indistinguishable from liquid culture controls. Moreover, the uncharged gels are expected to be highly insensitive to aqueous solvent composition. Together, these results lead to the selection of this microgel to be the main focus in later steps of the project. However, they have one major disadvantage; the uncharged gels require vigorous mechanical agitation to disperse and homogenize in liquid growth media. By contrast, the charged gels disperse very easily, requiring very little agitation. Refinements of the uncharged PEG formulation and further exploration of the potential of the least cytotoxic charged microgels will continue in future work to optimize the material for achieving the target liver microtissue performance.

Materials, Vendors and Part Numbers: PEGa 480 MW (Sigma Aldrich 454990); PEGda 700 MW (Sigma Aldrich 455008) Ammonium Persulfate (Sigma Aldrich A3678); TEMED (Fisher Scientific BP150); PGPR (Paalsgaard 4125); Kerosene (Sigma Aldrich 329460); Methanol (Fisher Scientific BPA413 20); Diethyl Ether (Fisher Scientific 615080040); 500 mL Centrifuge Tubes (Fisher Scientific 431123); 500 mL Centrifuge Tube Cushions (Fisher Scientific 431124); Ice; MilliQ Water.

TABLE 1
Examples of PEG micro-gel compositions

| Polymer | | | | |
|---|---|---|---|---|
| Concentration | PEGa | PEGda | APS | |
| (wt %) | 50% stock | 25% stock | 10% stock | Water |
| 25 wt % | 73.91 g | 2.18 g | 2.25 g | 71.66 g |
| 20 wt % | 59.13 g | 1.74 g | 2.25 g | 86.88 g |
| 15 wt % | 44.35 g | 1.31 g | 2.25 g | 102.10 g |

### Synthesis Protocol:

1. Create Organic Phase in 1L beaker
   a. Add 3.5 g PGPR to the beaker
   b. Add 500 mL Kerosene to the beaker
   c. Stir for 15 min
2. Create Aqueous Phase in 200 ml beaker
   a. Make 3 g of 10% ammonium persulfate solution
   b. Add PEGa to beaker based on desired concentration (see Table 1)
   c. Add PEGda to beaker based on desired concentration (see Table 1)
   d. Add 2.25 g ammonium persulfate
   e. Add Water based on desired concentration (see Table 1)
   f. Stir for 5 min
3. Combine organic and aqueous in 1L beaker
   a. Remove stir bar from organic solution
   b. Place beaker containing organic phase on ice
   c. Add in aqueous mixture
   d. Homogenize for approximately 5 min at 7000 rpm
4. Purge and stir solution
   a. Place solution in round bottom flask
   b. Put flask over ice and stir solution
   c. Purge with Nitrogen for 1 hour
5. Reaction of solution with TEMED
   a. Remove round bottom flask from ice
   b. Raise gas line from solution but still keep in flask to maintain nitrogen atmosphere
   c. Add 3 mL TEMED to the solution
   d. Stir for 1 hour
   e. Remove seal to allow air back in solution
   f. Stir for 30 min.
   g. Distribute evenly into eight 500 mL conical tubes
6. Perform methanol wash of gel
   a. Add methanol to make total volume in each conical tube approximately 500 mL
   b. Weight tubes to verify total weights are within +/−1% of each other
   c. Shake tubes vigorously for approximately 1 minute each
   d. Centrifuge at 4000 rpm for 30 minutes
   e. Remove supernatant
   f. Repeat
7. Collapse gels in ether
   a. Add diethyl ether to tubes to make total volume approximately 250 mL
   b. Weight tubes to verify total weights are within +/−1% of each other
   c. Shake tubes vigorously for approximately 1 minute each
   d. Centrifuge at 4000 rpm for 5 minutes
   e. Remove supernatant
   f. Repeat
8. Desiccate gel
   a. Distribute solution into approximately 15, 50 mL tubes with approximately 5 mL of gel placed in each.
   b. Desiccate for approximately 12 hours under vacuum

Charged poly(ethylene glycol) (PEG) microgels are synthesized through an inverse emulsion reaction; aqueous emulsion droplets containing poly(ethylene glycol) methyl ether acrylate (PEGa), charged comonomer (methacrylic acid (MAA), quaternized 2-(dimethylamino)ethyl methacrylate (qDMAEMA), carboxybetaine methacrylate (CBMA)), and poly(ethylene glycol) diacrylate (PEGda) are emulsified in a continuous organic phase, following the protocol described in Appendix 1 with the only modification being the addition of charged co-monomer. The compositions used to synthesize the charged PEG gels described in this report are given below in Table 2.

TABLE 2
Polymer concentrations for PEG microgels. Comonomers for charged microgels include
anionic (MAA), zwitterionic (CBMA), and cationic (qDMAEMA) comonomer.

| | | | | Charged Comonomer | | | |
|---|---|---|---|---|---|---|---|
| Polymer | PEGa | PEGda | APS | 17 mol % incorporation | | | |
| Concentration | 50 wt % | 25 wt % | 10 wt % | MAA | qDMAEMA | CBMA | |
| (wt %) | stock | stock | stock | anionic | cationic | zwitterionic | Water |
| 25 wt % | 73.91 g | 2.18 g | 2.25 g | 1.126 g | 3.904 g | 3.003 g | 71.66 g |

Supplies and part numbers for charged monomers: CBMA (M2359, TCI America); MAA (155721, Sigma Aldrich); DMAEMA (234907-100 mL, Sigma Aldrich).

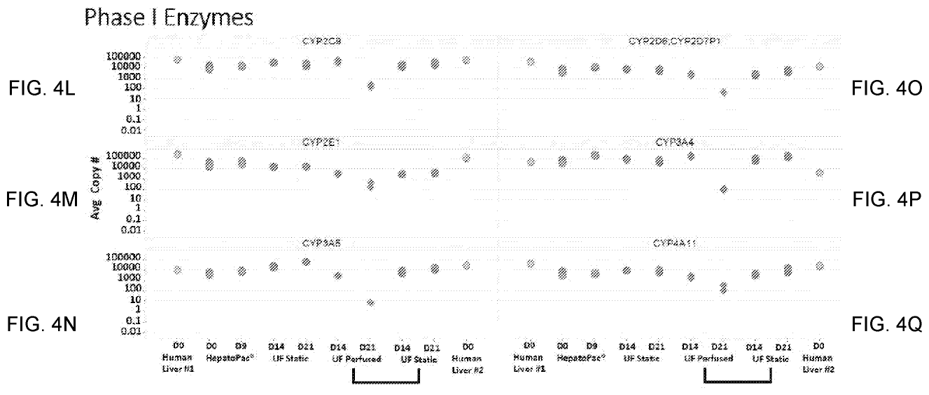

Quaternized 2-(dimethylamino)ethyl methacrylate (qDMAEMA) synthesis. Cationic microparticles are synthesized using quaternized 2-(dimethylamino)ethyl methacrylate (qDMAEMA). DMAEMA (18.7 g) is mixed in anhydrous THF (30 mL). Methyl iodide (20.2 g) in anhydrous THF (30 mL) is added dropwise at 0° C. The reaction mixture is warmed to room temperature and stirred for 24 h. The monomer is collected by vacuum filtration and rinsed on the filter with ethanol. NMR spectra is shown in FIG. 9.

### Example 3

A multi-material microtissue fabrication system is described. In terms of performance metrics, the functions assessed: standard deviation of microtissue size; mean size error; mean position error versus fabrication time. Together these demonstrate the precision & accuracy of liver microtissue biofabrication. The target specifications were: (1) relative error tolerance on positioning <50 microns per 1 mm of linear length; (2) standard deviation of longest linear dimension <5% relative to mean; (3) mean error of longest dimension <50 micrometers per 1 mm of linear length. In the results below, all these target specifications have been met.

### Results

### 1. Relative Error Tolerance on Positioning

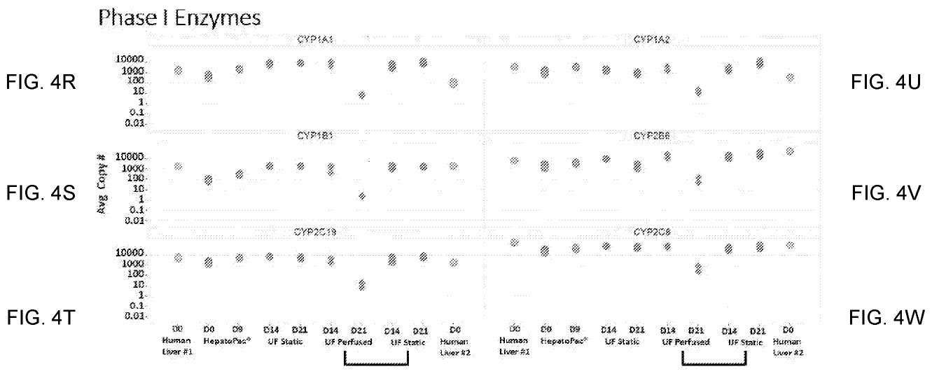

To investigate positioning errors with the DISC 3D bioprinter, a series of tests that established the uncertainty in measuring the location of the printing needle tip were performed, and then positioning errors that account for potential measurement inaccuracies determined. To establish the uncertainty in measuring the location of the needle-tip, the DISC printer was mounted atop an inverted fluorescence microscope (Nikon Ti-E) and collected images of a syringe needle tip held by the 3D bioprinter. To image the needle tip using fluorescence, micron-scale fluorospheres having red light emission were mixed with epoxy and the syringe needle filled, wiping excess epoxy away from the outside of the needle tip before curing. After curing the epoxy, the DISC control software was used to position the needle tip at five different locations within one field of view. At each position, video-rate images were collected for a few seconds, producing more than 100 images per location. The images were thresholded to determine the edge of the needle and the geometric center of the needle was computed from all needle edge locations. To measure the uncertainty in measuring a stationary object at a single location, the root-mean-square (RMS) deviation of the geometric centers was computed at each position within the field of view. To estimate an average uncertainty across measurements at different locations, the mean and standard deviation of the RMS deviations were computed across the five independent tests. The average RMS fluctuation in the x-position was Ex=0.19±0.10 μm (mean±standard deviation); the average RMS fluctuation in the y position was Ey=0.13±0.08 μm (FIG. 10). Thus, when positioning errors are computed in later tests, the contribution from random errors in measuring the needle location is expected to be less than 200 nm. To measure the error in positioning originating from the printer itself, 1 mm×1 mm square paths were created containing intermittent locations spaced by 100 μm where position measurements were made, moving at a translation speed of 0.3 mm/s between each move. Errors in positioning were computed and the test was repeated 3×. Throughout these tests, no single error measurement reached 20 μm, demonstrating that the target specification of <50 μm positioning error has been achieved. To analyze these positioning errors statistically, the RMS error within each single-square test was computed, then the mean and standard deviation of these errors across different tests was computed. The positioning error along the x-direction was Ex=6.1±4.2 μm; the positioning error along the y-direction was Ex=7.2±4.9 μm. Positioning errors are more than 30× larger than the largest measurement uncertainty, indicating that measurement uncertainty can be neglected. these measurements for moves performed at different translation speeds were not repeated as it has been found empirically that tissue quality does not exhibit systematic changes when printing within the range of 0.05 and 0.5 mm/s.

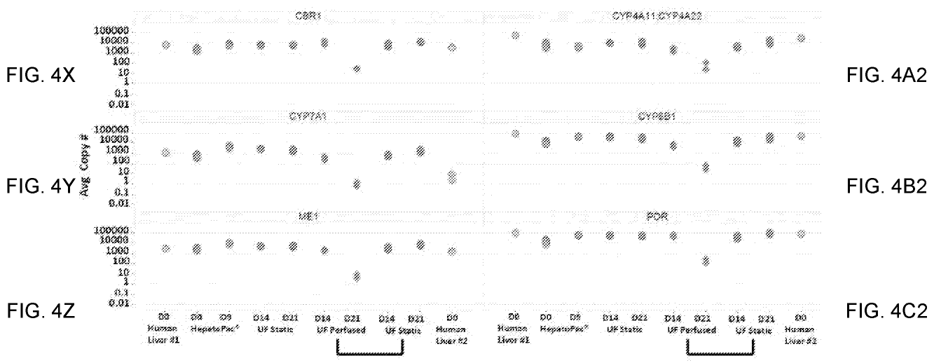

2. 3D Bioprinting and Data Analysis Details 3D bioprinting was performed using the DISC system, once again mounted atop an inverted microscope. The microgel-based 3D printing support medium was prepared as described in previous reports. Briefly, following synthesis and washing in PBS buffer, the microgels were equilibrated against an excess of cell growth media and transferred to tissue culture places where they were allowed to pack under gravitational forces. Excess liquid media was removed with a micropipette, and the well-plate was transferred to the stage of the microscope supporting the printer. Cells were harvested from culture plates and mixed with solutions of collagen-1 to provide an extracellular matrix (ECM) microenvironment that promotes tissue cohesion. This solution is loaded into a syringe which is then mounted onto the DISC bioprinter. Before printing, the needle tip was located using the crossed laser-sensor system on the DISC printer. During the prints, the needles were translated at 0.3 mm/s and the material deposition rate was set to 50 μL/h. Following printing, the well plates were transferred to a laser-scanning confocal microscope (Nikon C2+) to image the printed tissues (FIG. 11).

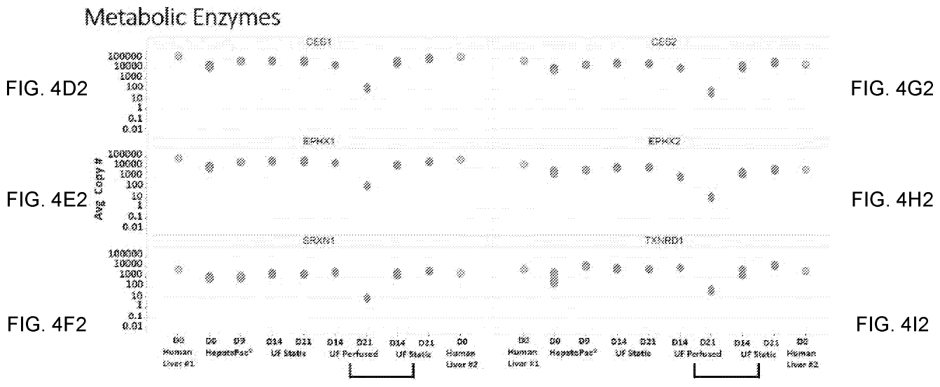

To eliminate seams, corners, and potential stagnation points where material may accumulate, structures are bioprinted in single, smooth spiral paths. Additionally, disk-shaped structures are printed that span millimeter scales laterally but are approximately 200 μm thick. These combined strategies enable creating highly-controlled structures made from large numbers of cells that are not expected to exhibit rapid necrosis anywhere within the tissue associated with potential hypoxia. a spiral pitch of 100 microns was chosen, which seals features together between adjacent passes around the path. (FIG. 12).

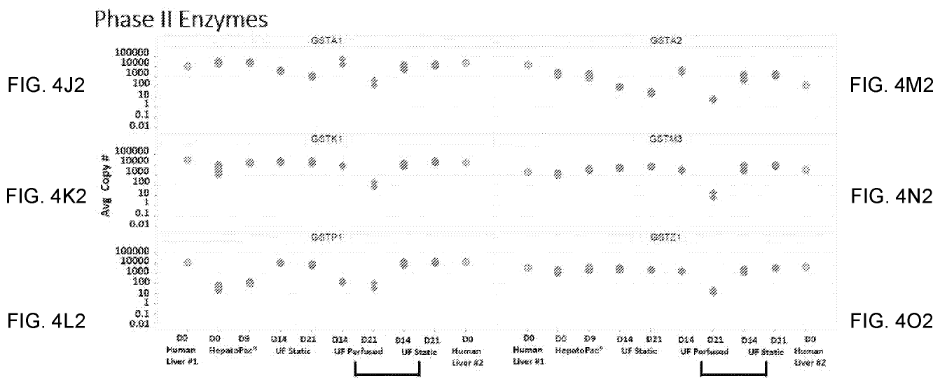

To determine the length of the largest dimension in the printed tissue, d, image processing on the 3D stacks collected on the confocal microscope was perform. Maximum-intensity projections are performed along the thin-axis of the disk-shaped tissues, and the projected images are thresholded. A convex hull is traced around the tissue image and the area is computed. A formula was derived that relates this area to the effective diameter, d, of the spiral, which is the distance between the outermost point in the spiral to the opposing surface, accounting for the spiral asymmetry and the width of the features. Converting measured area to d, the length of the longest feature for each tissue was determine. This procedure reduced the contributions of spurious features that artificially increase measured variability when using other methods (FIG. 13).

### 3. Standard Deviation of Longest Linear Dimension; Mean Error of Longest Dimension

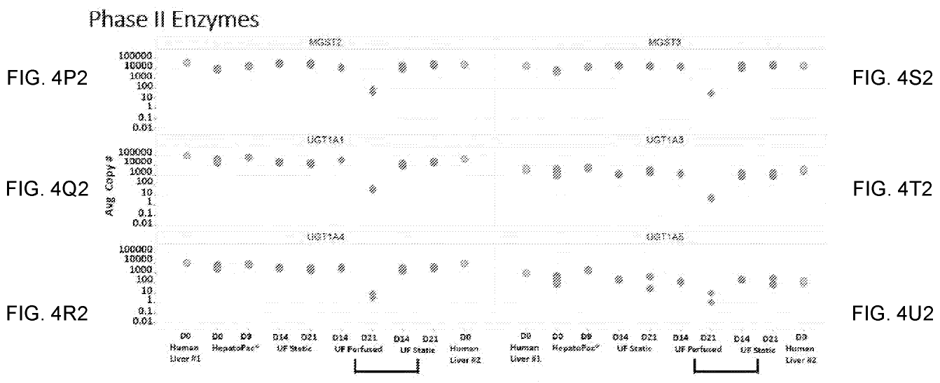

To measure the standard deviation of the longest linear dimension, dm, of 3D printed tissues, tissues programmed to exhibit expected diameters were created, de, of 1.1 mm, 2.2 mm, and 3.3 mm. In these tests, the tissues were made from HepaRG hepatocytes, mixed with collagen-1 at a final concentration of 0.5 mg/mL before printing. Processing the 3D confocal stacks, dm for each individual tissue was determined and replicate experiments for each designed size were performed. The % error in dm for each experiment across all programmed size-scales, relative to the expected diameter, de was computed. The standard deviation of these % errors were determined to be 0.82%, much less than the target value of 5%. The average % error was found to be 4.2%, with a maximum single-measurement error of 10.9%. The absolute error per mm of linear length, averaged across all these measurements was 42±3.0 μm. Thus, on average it was possible to repeatedly print structures having diameters 3.3 mm or less within the target accuracy of 50 μm per mm of linear length. The average error per mm of length for the 1.1 mm diameter tissues, in absolute units of mm, was 25 μm; for the 2.2 mm diameter tissues it was 46 μm; for the 3.3 mm diameter tissues it was 55 μm. In the case of the de=3.3 mm tissues, measurements were not performed to determine the uncertainties of determining dm from the methods described here, the measurement uncertainty is expect to be greater than the 5 μm difference between 55 μm and 50 μm. Thus, these tissues likely satisfy the target specifications within their own group (FIG. 14).

### 4. Multi-Material Printing Capabilities

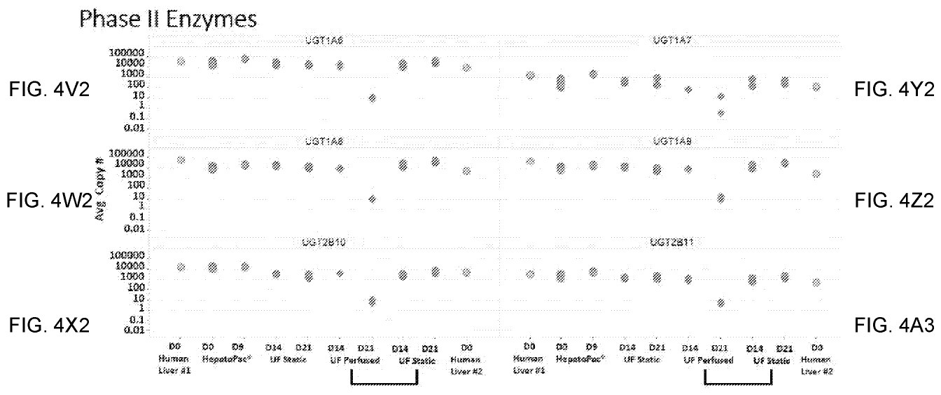

To test and demonstrate the multi-material functionality of the DISC 3D bioprinter, two-layer tissues were created from hepatocytes and cholangiocytes. Cells were cultured in standard flasks and microgel-based support medium was prepared as previously described using William's E media with standard supplements. Cholagiocytes were dyed with CellMask Red (ThermoFisher) and hepatocytes were dyed with CMFDA (Thermofisher), a green cytosol dye. Cells were harvested from the flasks, concentrated into pellets at the bottom of 0.5 mL micro-centrifuge tubes, and manually re-dispersed with manual pipetting. The micro-centrifuge tubes containing the cells were transferred to the well-plate inserts designed by DISC (Appendix A). The inserts were placed in empty wells of the well plate where printing was performed; the microgel media was deposited into adjacent wells. The DISC printer was programmed to collect cholangiocytes (FIG. 154, red) by positioning the needle tip near the bottom of the corresponding centrifuge tube and raising the plunger. The syringe containing the cells was then moved to the print wells, where printing was commenced, as previously described. After printing discs made from cholangiocytes, the syringe was moved to the centrifuge tube containing the hepatocytes, where it collected more cells. The hepatocyte-filled syringe then returned to the wells where it previously deposited cholangiocytes, and created new hepatocyte layers. Prints were programmed to have varied spacing between the two cell layers, starting at zero-separation in which the cells were expected to mix. Examining these different samples on the confocal microscope revealed that the two cell layers could be positioned with controlled spacing. The sample in which no spacing between the layers was programmed exhibited overlap and mixing of the two cell populations. With increasing programmed spacing, the separation between the layers was found to increase systematically (FIG. 15), demonstrating the multi-material capabilities of the DISC printer.

### Example 4

Development and evaluation of new protocols to optimize measurements of Albumin and Urea within the LLS. New protocols will include adaptation of ELISA assays in which reagents are mixed with LLS media.

### Overview and Principle Conclusions

ELISA and colorimetric-based chemical assays were developed for quantitative detection of albumin and urea in hepatocyte cell cultures with modifications to manufacturers' protocols. The assays were developed to enable monitoring of hepatocyte cell cultures over time, and to investigate parameters for improved model liver tissue function, including comparison of different hepatocyte sources, effects of co-culture (heterotypic cell interactions) and perfusion in 3D printed structures. The data show that PEG microgels can be sampled directly for assay measurements but require further dilution (in media or assay buffer) to 2.25% or lower, for albumin, and 0.9% or lower, for urea to ensure accuracy. Secreted urea and albumin synthesized by cultured hepatocytes uniformly distribute within the microgel and surrounding media, enabling the possibility of repeated media sampling (of supernatants or flow-through) in 3D printed cultures for monitoring of synthesis levels in longitudinal studies. To meet the required stage 1 performance targets and specifications, a DNA content assay was also developed using PicoGreen for estimation of cell numbers in bioprinted cultures recovered from microgel media. The data presented show cell number estimations of 3D printed spheroids and discs, and the effects of microgel type and perfusion culture. The cell number calculations will be used to normalize daily production rates (μg/day/106 hepatocytes) for comparison to specified target values in future studies.

The work described in this report also provides initial characterization of albumin gene reporter vectors for cellular-level assessments of hepatocyte function in 3D culture. Lentiviral-based vectors encoding fluorescent proteins were generated and demonstrated preliminary evidence of liver-specific reporter expression in HepG2/C3A cells. Following their further validation, the reporter vectors may be used to in place of protein assays for optimization of liver microtissue biofabrication and non-destructive quality control monitoring.

### Results

### 1. Optimization of Albumin and Urea Assays for Detection in 3D Microgel Cultures

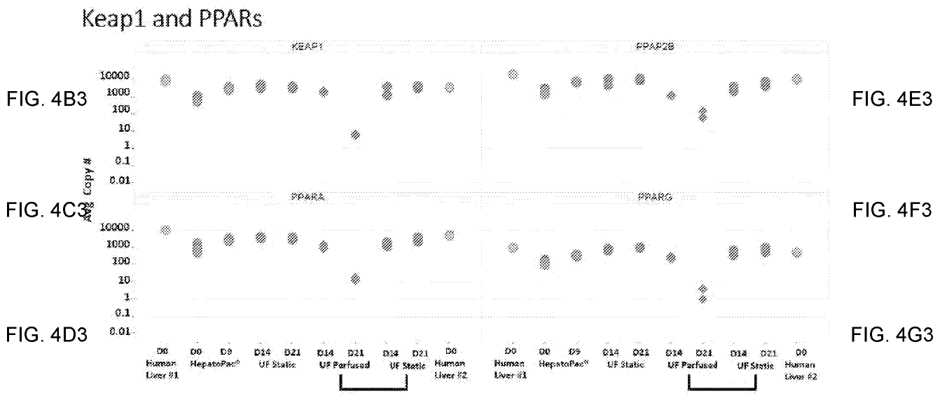

Albumin-Albumin levels were measured in liquid culture medium and PEG microgel media by sandwich ELISA using the Human Albumin ELISA kit (Cat #E88-129, Bethyl Laboratories, Montgomery, Texas), according to the manufacturer's protocol (Appendix 1). Initial tests were performed to determine FIG. 16A shows the standard curves of albumin (0-167 ng/ml) prepared in assay buffer (supplied by the manufacturer), growth (HepaRG) medium, or a range of microgel concentrations (0.45-4.5% PEG, swollen in HepaRG medium). Regression analysis (4-parameter fit) revealed R2 values of ≥0.99 for media, assay buffer and 0.45-2.25% PEG microgels, suggesting minimum interference of microgels at these concentrations. However, there was a marked decrease in absorbance with 4.5% PEG for albumin concentrations >˜10 ng/ml, suggesting impairment of protein binding. Based on these findings, dilutions of sampled PEG microgels to ≤2.25% would be required for accurate determination of albumin synthesis levels by ELISA.

Urea-Urea levels in liquid media and PEG microgel culture samples were initially measured using the QuantiChrom Urea Assay Kit (Bioassay Systems, Hayward California). Using this method, absorbance at 520 nm for colormetric measurement of urea complexes was found to be unsuitable for samples, due in part to the presence of phenol red in hepatocyte growth medium. Additionally, the growth media was found to react with QuantiChrom reagents in the absence of urea, leading to increased absorbance over the assay duration. As a result of this background interference, measurements of low urea concentrations (˜0.78-˜ 12.5 μg/dL) had a predicted error of ˜30-˜ 230% (data not shown) and thus could not be accurately determined. The Stanbio Urea Nitrogen (BUN) kit (Stanbio Labs, Boerne, Tx) utilizes a spectrophotometric method based on the reaction of urea with diacetyl monoxime in highly acidic conditions. Under the low pH of the reaction (<4) phenol red does not absorb at 520 nm eliminating the possibility of background signal from the media. A modification of the assay protocol developed for a 96-well plate format (Appendix 2), was used to investigate the sensitivity of urea detection in liquid culture media and PEG microgel samples. Urea standards were generated (0.78-50 μg/dL) in hepatocyte liquid growth medium and PEG microgels of varying concentration as described for FIG. 1a. Absorbance was found to be linear for all urea concentrations tested, with R2 values of >0.97 (linear curve fit) in liquid growth media and all microgel dilutions (FIG. 16B). As with albumin, there was a notable reduction in overall absorbance with 4.5% PEG, and to a lesser degree, 2.25% PEG, while concentrations of 0.9% and lower were indistinguishable from media controls. From these data, it was determined that PEG microgels can be sampled and assayed for accurate determination of urea concentrations following dilution to 0.9% or lower.

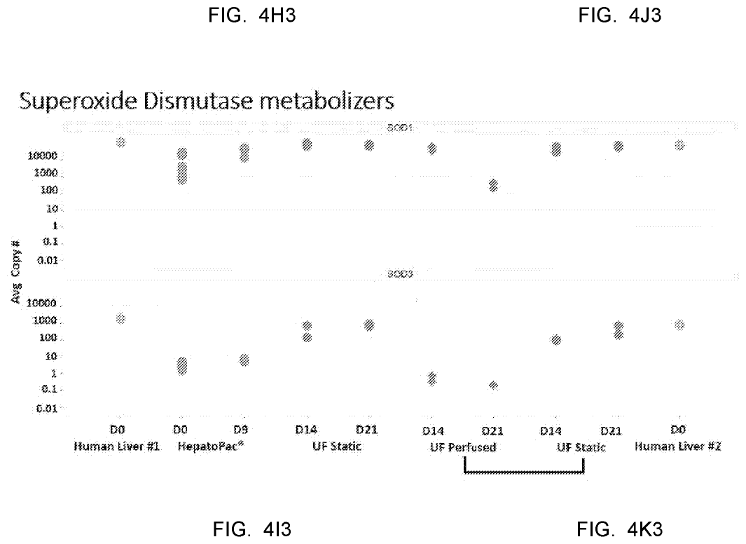

Assay modification for urea measurements in 3D printed cultures—The capacity for detection of urea concentrations in hepatocyte cell cultures with low synthesis levels may be limited by the requirement of a large sample dilution (10 μL in 180 μL of assay buffer) in the BUN assay protocol. This is further compounded with the use of large culture volumes that incorporate both microgel and growth media phases in perfusion-based systems. To increase assay sensitivity, the volume of sample was increased relative to assay buffer to determine if linearity was maintained over a range of urea concentrations. FIG. 17A shows that over the range of urea concentrations tested, linearity was maintained when sample volumes were increased 3- and 5-fold, with R2 values >0.97. Regression analysis of replotted data over a lower urea concentration range (0-12.5 μg/dL) further revealed increased sensitivity and linearity when 30 μL and 50 μL sample volumes were used (FIG. 17B) with R2 values >0.99, compared to 0.76 using the standard protocol (10 μL). Thus, increasing sample volumes above 10 μL led to enhanced assay sensitivity. Based on these results, the assay protocol has been modified for analysis of 30 μL sample volumes to provide greater assay sensitivity in recovered media samples.

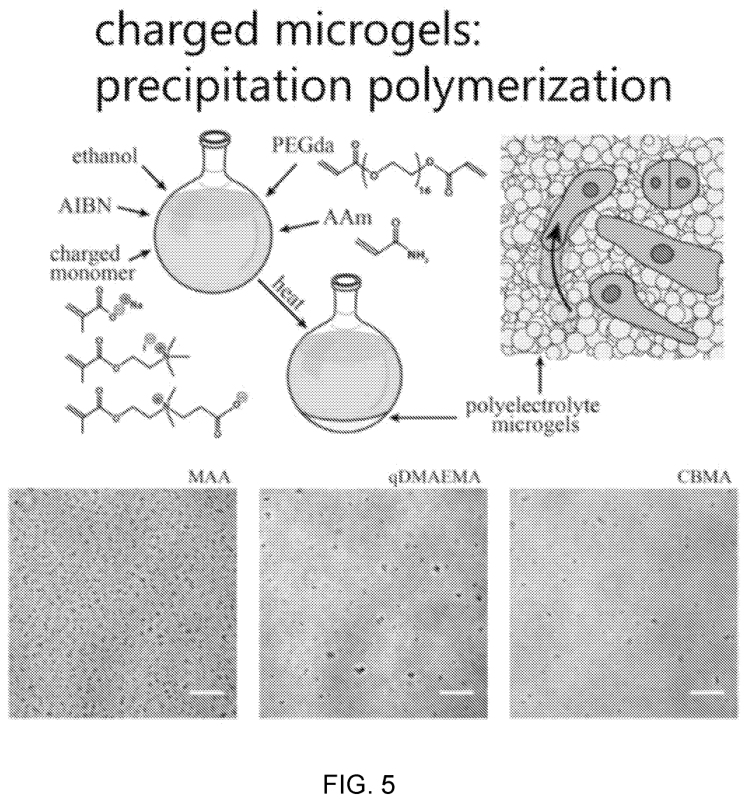

Albumin and urea distribution—The polymer mesh and pore space in the microgel medium is large enough to allow unimpeded diffusion of large proteins and small molecules. Therefore, synthesized liver proteins in microgel cultures were expected distribute evenly throughout the microgel media and surrounding liquid growth medium. To investigate albumin and urea distribution within gel and media phases in cell culture, confluent monolayers of hepatocytes (HepaRGs) were overlaid with equal volumes of microgel (4.5% PEG), and HepaRG growth medium and cultured for 72 h. Microgel media and liquid media were then sampled and assayed for albumin and urea levels by ELISA and BUN assay, respectively (FIGS. 18A-18C). Albumin concentrations were approximately equal (˜2.5 μg/10⁶ cells) in both gel and media phases, and matched synthesis levels in control wells containing media only (FIG. 18A). Likewise, urea concentrations were similar in both media and gel phases (˜0.5 μg/10⁶ cells), and comparable to media only controls (FIG. 18B). Together these data indicate that secreted albumin and urea by hepatocyte cultures does not preferentially accumulate within the PEG microgel, but instead reaches uniform concentration in both gel and media phases. Cell supernatants or flow-through media in printed microgel cultures can therefore be readily accessed for sampling to obtain representative measurements of albumin and urea production with minimum disturbance of printed structures within the microgels.

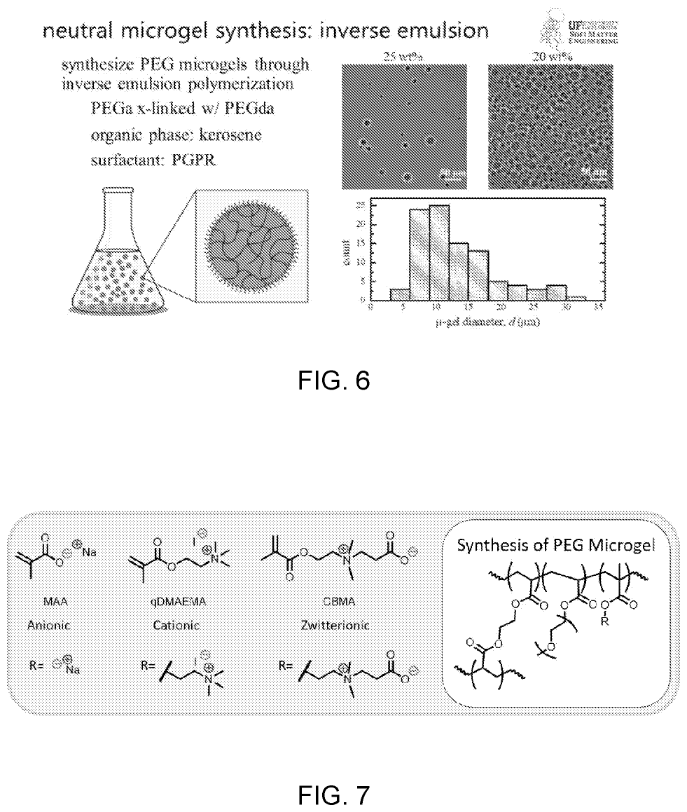

Cell number determination from 3D printed structures-Cell number estimation was performed on cell cultures recovered from microgel media using a modification of the PicoGreen dsDNA quantitation assay (Molecular Probes, Eugene, OR), adapted for analysis of 3D microtissues (Appendix 3). Printed spheres or discs were harvested from the microgel media, washed extensively in PBS to remove soluble DNA fragments, and lysed to release genomic DNA by papain digestion and freeze-thaw. Aliquots were then incubated with PicoGreen and measured using a fluorescence plate reader. Standard curves were generated to establish the dynamic range and sensitivity of fluorescence vs DNA content using a calf thymus DNA standard (10-1000 ng/ml; FIG. 19A) or vs cell count using fixed numbers of pre-cultured cells (10³-10⁶ cells; FIG. 19B). FIG. 19C shows the estimated cell numbers in printed 1.5 mm diameter spheroids of MG63 (a tumorigenic, osteosarcoma cell line) cultured in 1.5% polyacrylamide (pAAm) or 4.5% PEG microgels. Calculated cell numbers were approximately 250,000 to 300,000, which was comparable with non-printed spheroids (approximately 1-1.5 mm diameter) generated by centrifugation (typically 250,000 cells, data not shown). Roughly equivalent cell numbers were determined at two time points post-print (days 5 and 8), indicating similar cell viabilities of MG63 in pAAm and PEG microgels (FIG. 19C).

Preliminary assessments of hepatocyte cell viability following printing into microgel media were also investigated under static (no flow) and perfusion conditions (FIG. 19D). HepaRG cells were mixed with MatriGel and printed as 2 mm diameter discs into 4.5% PEG microgel media; the microgel media was contained in well-plates equipped with custom perfusion chambers that permit delivery, continuous exchange, and collection of liquid growth media. Calculated cell numbers at 1- and 7-days post-print revealed ˜100,000 cells under both static and perfusion conditions, in line with the predicted cell number based on initial cell concentration prior to printing. These findings suggest that the absence of fluid exchange in PEG microgels does not adversely affect the viability of HepaRG cells when maintained as thin layered disc structures. This work is currently being extended to investigate the relationship between cell viability and various culture parameters to examine the influence of matrix, cell density, effects of coculture, and extended culture duration.

### 2. Characterization of Albumin Gene Reporters for Monitoring Hepatocyte Function in 3D

Protein assays and molecular profiling are useful quantitative measures for monitoring liver-specific functions in 3D cultures, but they are often restricted to endpoint analysis and do not distinguish cellular-level responses. Gene reporter constructs that encode fluorescent proteins under conditional activation of tissue-specific promoters can provide valuable tools for quantitative and spatial assessment of cellular-level function in real-time. This tool will enable non-destructive quality control tests to be performed on liver model tissues before shipping. To facilitate liver microtissue fabrication using the microgel-based bioprinting platform, lentiviral reporter vectors have been constructed encoding fluorescent proteins in an effort to identify cells with enhanced hepatogenic function. The vectors contain dual independent expression cassettes encoding fluorescent reporter genes under control of 1) a constitutive promoter (EF1α), where reporter expression serves as an index of the viable population of transduced cells, and 2) a hepatocyte lineage-specific promoter (Alb), derived from regulatory sequences of human ALB, whose activation signals hepatocyte specificity [M. Fran et al., Mol Cell Biol 10:991-99 (1990); J. Tang et al., Biomed. Rep. 6:627-32 (2107)].

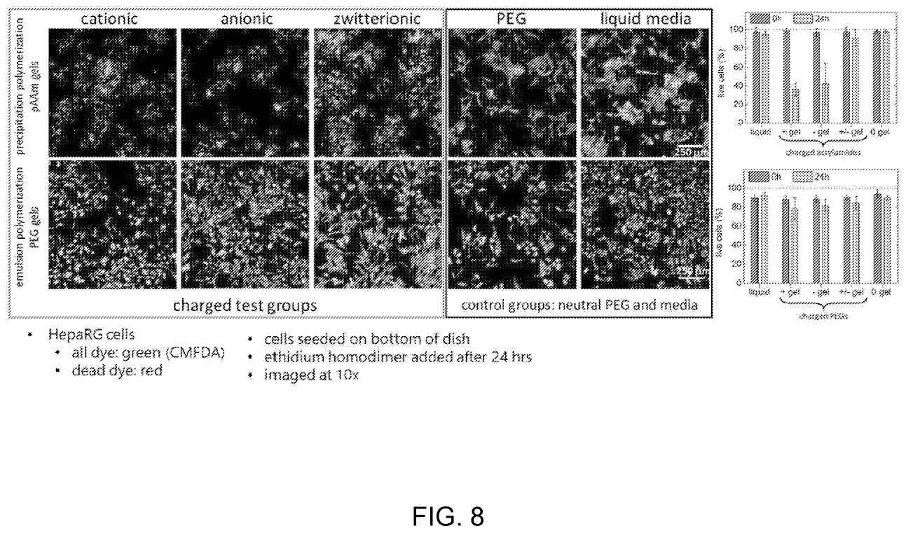

DNA sequences encompassing the albumin core promoter were synthesized and cloned into lentiviral vector plasmids, with and without an upstream enhancer element, previously shown to regulate albumin transcriptional activity [M. Fran et al., Mol Cell Biol 10:991-99 (1990)]. Recombinant lentiviral vectors were generated following transfection in 293 cells, which contained GFP under regulation of a constitutive EF-1 promoter, and td-tomato (a red fluorescent protein) under conditional activation of the proximal albumin promoter (pAlb) or albumin promoter/enhancer (pEAlb) (FIG. 20).

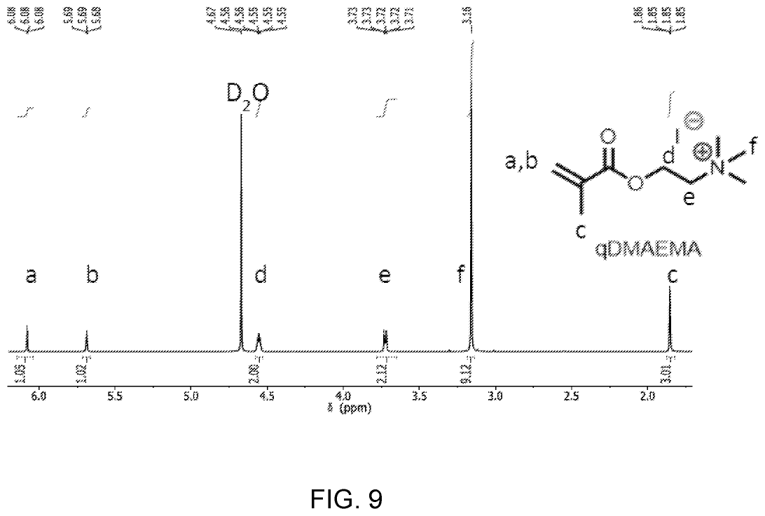

To test the sensitivity and specificity of the reporters, red and green fluorescence were examined following lentiviral transduction of the hepatocyte cell line, HepG2/C3A, and the osteosarcoma cell line MG63, serving as a non-hepatocyte cell control. High transfection efficiency, indicated by abundant GFP fluorescence, was evident for both reporter vectors in both cell types (FIGS. 21A-21H, panels a,b,d,e). In HepG2/C3A cells, a subpopulation of GFP+/tdTom+ (green+red) could clearly be seen with both reporter vectors (FIGS. 21A-21H, panels a-c), indicating activation of pAlb and pEAlb. In contrast, GFP+/tdTom+ cells were not detected for either vector in MG63 cells (FIGS. 21A-21H, panels d-f) indicating that the albumin promoters were not active in this cell line. Cell quantification revealed a low (˜2-3%) frequency of GFP+/tdTom+ cells using both pAlb and pEAlb, suggesting similar sensitivity of both reporter vectors in HepG2/C3A cells (FIGS. 21A-21H, panels g,h).

These findings provide validation of albumin gene reporter constructs for cellular-level assessment of liver-specific functions in hepatocyte cell lines.

### Appendix 1:

### Basic Overview of Provided Protocol:

1. Add 100 μL of standard or sample to designated wells.

Note: Run each standard or sample in duplicate.
2. Cover the plate and incubate at room temperature (20-25° C.) for 1 hour.
3. Wash the plate FOUR times.
4. Add 100 μL of anti-albumin Detection Antibody to each well.
5. Cover the plate and incubate at room temperature for 1 hour.
6. Wash the plate FOUR times.
7. Add 100 UL of HRP Solution A to each well.
8. Cover the plate and incubate at room temperature for 30 minutes.
9. Wash the plate FOUR times.
10. Add 100 μL of TMB Substrate Solution to each well.
11. Incubate the plate in the dark at room temperature for 30 minutes.
12. Stop the reaction by adding 100 μl of Stop Solution to each well.
13. Measure absorbance on a plate reader at 450 nm

### Appendix 2:

The protocol for assessing urea was as follows:

Function Assessment: Urea Nitrogen (BUN) Test (Stanbio, cat #: 0580-250)
1. Make 7 standards by using the kit provided stock standard 250 μg/mL (=25 mg/dL) dilute it with your assay media to 50 μg/mL, 25 μg/mL, 12.5 μg/mL, 6.25 μg/mL, 3.125 μg/mL, 1.56 μg/mL and 0.78 μg/mL in 1.5 mL tubes
2. Add 160 μL Maintenance medium to 50 μg/mL standard tube, add 100 μL Maintenance medium to rest of 6 standard tubes
3. Add 40 μL 25 mg/dl standard to 50 μg/mL standard tube, mix well, transfer 100 μL 50 μg/mL to 25 μg/mL standard tube for the 1:2 dilution, and continue for rest standard tubes
4. Each standard will be run triplicate and medium samples will be run duplicate in 96-well PCR plate
5. Pipette 30 μL of standard and medium sample to the 96-well PCR plate (*Alteration from initial protocol to allow for better detection at lower concentrations)
6. Mix BUN reagent
   Determine the volume of BUN reagent necessary for 150 μL per well
   The BUN reagent consists of one part “color” solution and two parts “Acid” solution
   Add the appropriate volumes reagents to a 50 ml tube and mix thoroughly
7. Transfer the mixed BUN reagent into a pipette basin
8. Add 150 μL of BUN reagent to each well of standards and samples
9. Seal the PCR plate, mix, quick spin
10. Incubate the plate in GeneAmp PCR system 9700 in 99.9C for 13.5 minutes, then, 4C for 5 minutes to stop reaction. (the color will be stable for 30 minutes)
11. Quickly spin down
12. Transfer all solution from the PCR plate to 96-well Costar 9017 plate for the reading the signal by Paradigm
13. To be sure to turn on Paradigm (plate reader) at least 30 minutes before incubation is completed
14. Plate are read at \1=540 nM and A2=650 nM and reduction as!Lm1-! Lm2
15. Save the instrument data in ILS drive
16. Repeat steps 1 to 14 for additional plates to be read
17. Relevant instrument acquisition and data analysis parameters can be found in the respective *. sda data files stored in OpenLAB.
18. Data analysis:
   Unknown values are calculated from a liner standard curve of O.D vs. known standard concentration
   Linearity is impartment for accurate quantification of unknowns
   R2 value for the standard curve should be >0.95

### Appendix 3:

For determination of DNA content (and cell number) in 3D printed cultures the cell constructs are first removed from microgel media and carefully washed in PBS to remove protein/DNA fragments from lysed apoptotic/necrotic cells. The cells are then digested with papain and lysed by freeze-thaw. The cell digests are then diluted with TE buffer for assayed for DNA content by incubation with PicoGreen.

### Pellet Digestion Reagents:

GAG/DNA dilution buffer:
   50 mM sodium phosphate (e.g., Sigma #S9638)
   5 mM ethylenediaminetetraacetic acid (EDTA) (e.g., Sigma #E5134)
   2 mM dithiotreitol (DTT) (e.g., Sigma #D5545)
   Adjust pH to 6.8 using 1M NaOH
Papain digestion solution (300 0 g/mL):
   For every 100 mL solution, dissolve 30 mg papain (Sigma #P4762) in 100 mL GAG/DNA dilution buffer

### DNA Assay Reagents:

Black 96-Well polypropylene assay plates (Corning #3356)
Quant-iT™ PicoGreen® dsDNA Assay Kit (Molecular Probes #P7589), which includes:
   Quant-iT™ PicoGreen® dsDNA reagent (Molecular Probes #P7581)
   20× Trypsin-EDTA (TE) Buffer (200 mM Tris-HCl, 20 mM EDTA, pH 7.5)
   DNA stock solution (100 μg/mL in 1× TE Buffer)
DNA working solution (400 μL, sufficient for making duplicate standards):
   8 μL of 100 μg/mL DNA stock
   392 μL of 10 TE buffer (10 mM Tris-HCl, 1 mM EDTA, pH 7.5)
PicoGreen working solution (2×100 μL/well for X samples+8 standards+1 extra volume):
   Prepare sufficient volume of 1× TE buffer=(X+9)×200 μL
   Dilute PicoGreen reagent stock 200-fold in 1× TE buffer
   Protect from light

### Pellet Digestion:

1. Prepare 0.5 mL tubes with 200 IL papain digestion solution.
2. Harvest or aspirate culture medium and rinse pellets once with PBS. Aspirate wash volume.
3. Optional: Capture digital photograph of pellets in well plate over black background.
4. Transfer pellets to individual 0.5 mL tubes containing digestion solution.
5. Transfer tubes to a heat block or incubator set to 60° C. and digest pellets overnight. If no agitation is provided, vortex tubes once during incubation to help dissociate pellet.
6. After digestion, take tubes through 3 freeze-thaw cycles in order to ensure release of genomic DNA from dissociated cells.

### DNA Assay (PicoGreen):

1. Prepare 400 μL of 2 μg/mL DNA working solution by mixing 8 μL of DNA stock solution (100 μg/mL) and 392 μL of 1× TE buffer.
2. Prepare the following DNA standards using the 2 μg/mL working solution and 1× TE buffer:

TABLE 3
Standards Concentrations

| Conc. DNA | DNA Working | 1× TE buffer |
|---|---|---|
| ng/ml | Sol'n (μL) | (μL) |
| 0 | 0 | 250 |
| 10 | 1.25 | 248.75 |
| 50 | 6.25 | 243.75 |
| 100 | 12.5 | 237.5 |
| 200 | 25 | 225 |
| 400 | 50 | 200 |
| 800 | 100 | 150 |
| 1000 | 125 | 125 |

3. Prepare samples by diluting a 50 μL aliquot of each in 200 μL 1× TE buffer.
4. Add duplicate volumes (100 μL/well) of standards or diluted samples to a black 96-well assay plate.
5. Add 100 μL/well of PicoGreen working solution to the assay plate and protect from light.
6. After a 5 min incubation, measure the samples on a fluorescence microplate reader (excitation=485 nm, emission=538 nm).

### Example 5

### Executive Summary

This example evaluated liver enzymatic function, focusing on the following selected enzymes: CYP1A2, CYP2B6, CYP2C9, CYP2D6, CYP3A4 and UGT1A1.

These assays demonstrate metabolite formation related to all these enzymes.

### Overview and Principal Conclusions

Liver microtissue models were fabricated and incubated static culture conditions for 15 days, manually exchanging 0.3 mL of media every 4th day, or under perfusion with 1.0 mL of media exchanged daily. The metrics assessed were the detection of:

### Oxidized Nifedipine (CYP3A4), Dextrorphan (CYP2D6), Hydroxydiclofenac (CYP2C9), Acetominophen (CYP1A2), Hydroxybupropion (CYP2B6), 7-Hydroxycoumarin Glucuronide (UGT1A1).

### Results

To assess enzyme function of the 3D prints, a probe substrate cocktail was used to monitor metabolite formation related to key enzymes involved in small molecule metabolism. The assessment focused on the following enzymes: CYP1A2, CYP2B6, CYP2C9, CYP2D6, CYP3A4 and UGT1A1. Substrate concentrations were based on historical data and have demonstrated linear formation kinetics across the specified time course. The rate of metabolite formation was used to elucidate functional enzyme activity in the 3D liver system.

The initial assessment investigated two separate substrate incubation techniques (microtissue removed and substrate incubated in Eppendorf tubes vs direct substate addition to microtissues in gel/well) and the effect of perfusion vs static culture. Only direct substrate addition to microtissues under static conditions provided consistently measurable metabolites during the time course and were operating under linear kinetics. The incubation time for the perfusion samples was insufficient to capture any metabolic activity likely due to under-estimation of substrate diffusion to the hepatocytes. Comparing the static incubations for CYP2C9, the in well with hepatocyte cell suspension controls appeared to demonstrate a higher rate of metabolism. All other enzymes showed similar rates across the time course.

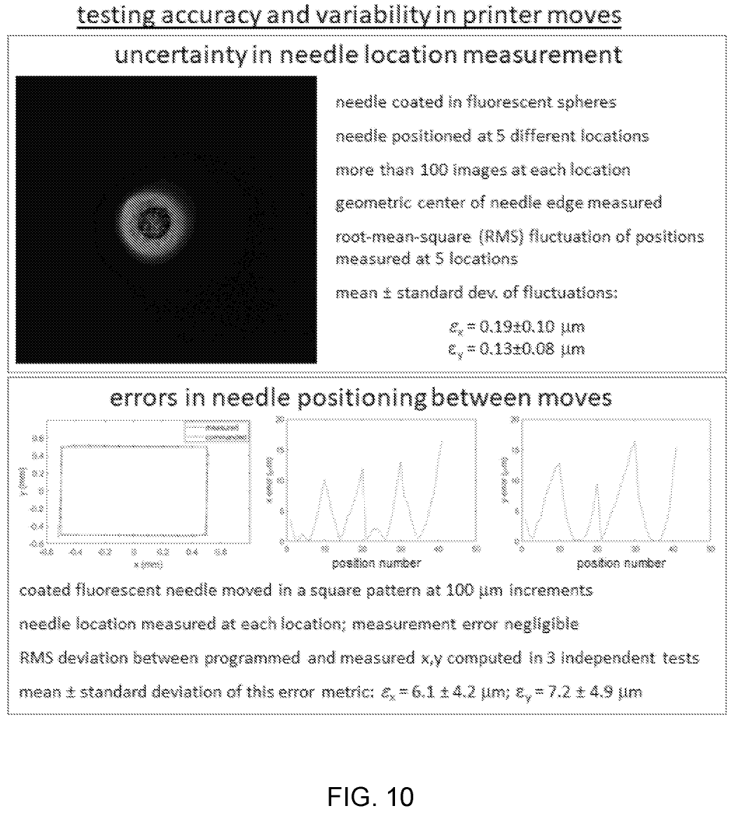

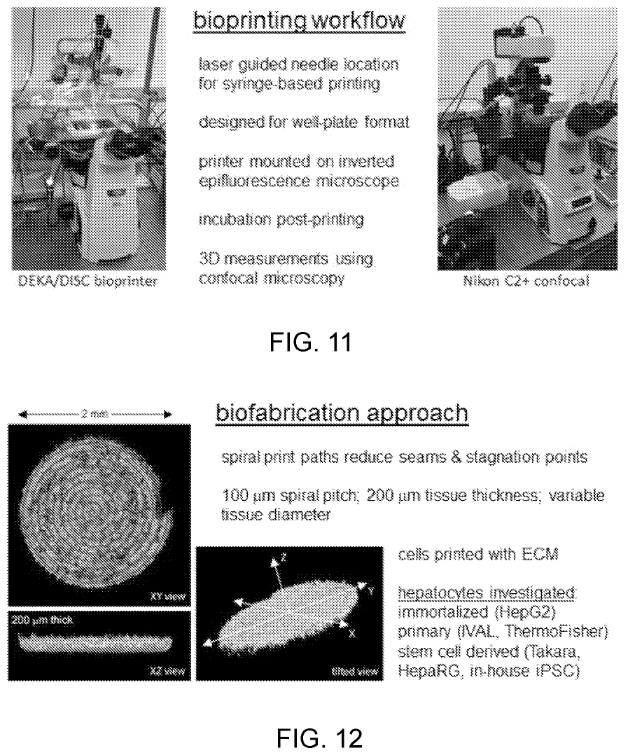

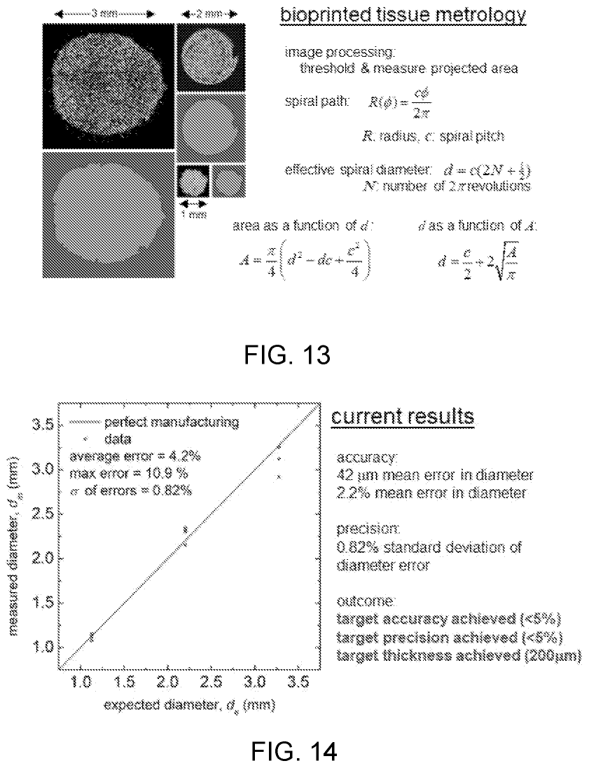

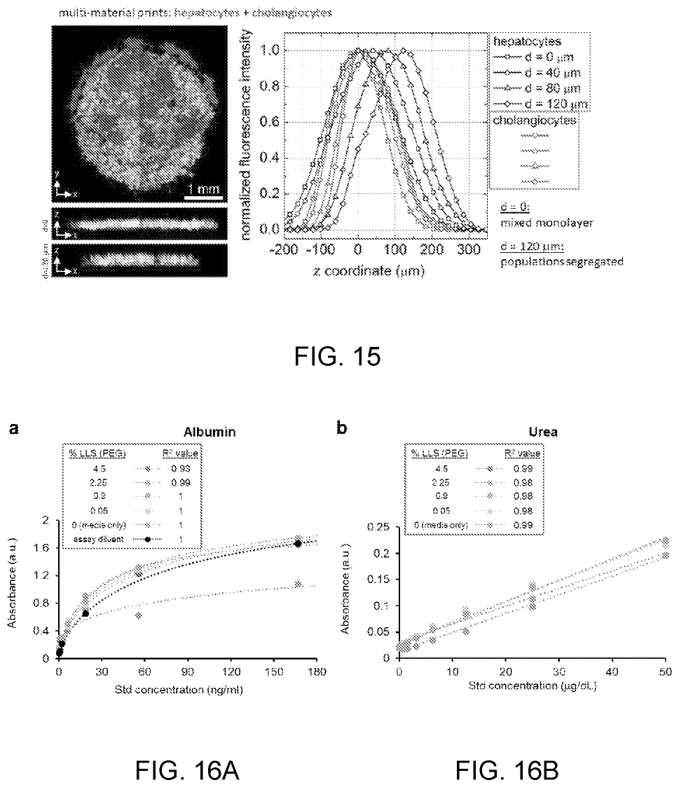

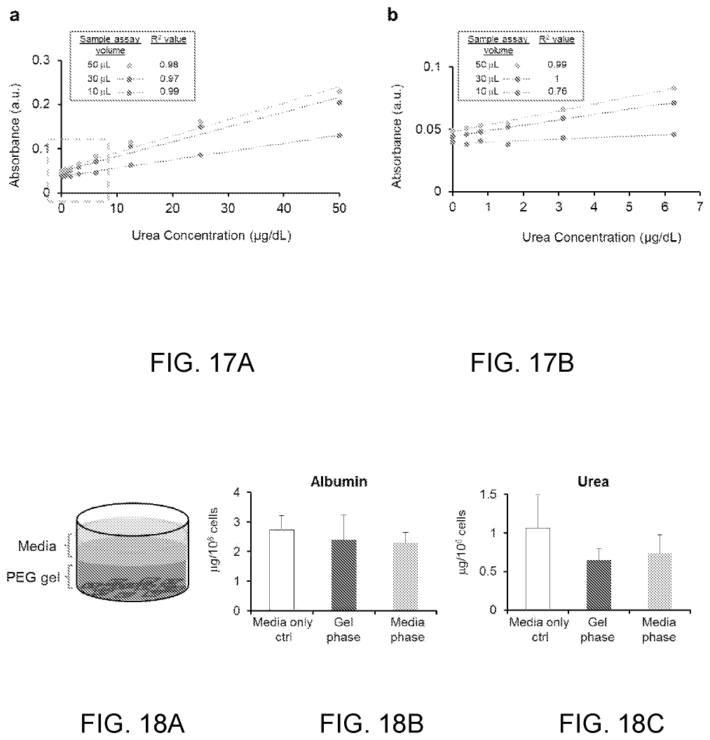

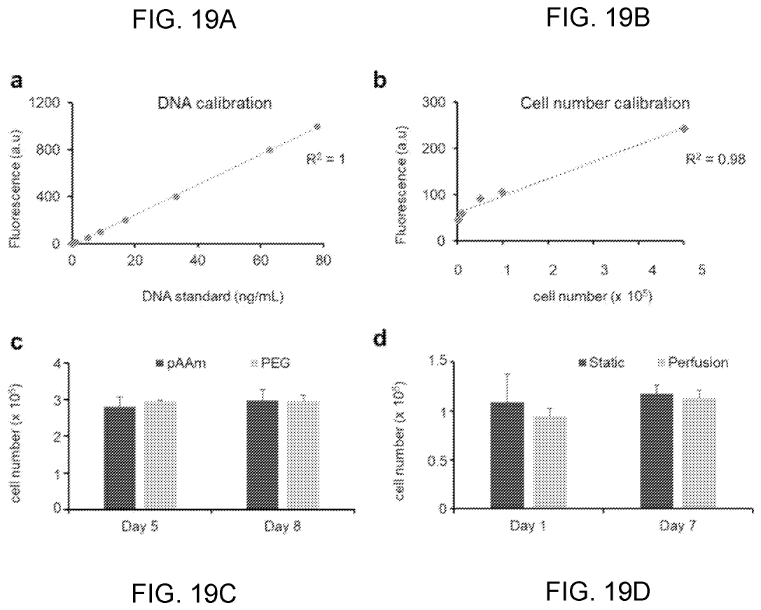

Figures Summary: Liquid chromatography-mass spectrometry (LC-MS) peak ratios are plotted versus metabolite formation time after the addition of substrate (FIGS. 22-27). The increase in peak ratio as a function of time indicate metabolite formation. Different substrate addition protocols were tested, producing comparable results for most enzymes tested.

### Appendix: Methods

Cell culture and microtissue generation: Hepatocyte metabolite function in 3D culture was evaluated for co-cultures of commercially available human, donor-derived primary cells (Thermofisher; Cat. #HMCPTS), with human umbilical vein endothelial cells (HUVEC; Lonza, Cat #C2519S). For microtissue generation, cell suspensions were obtained directly from thawed, cryopreserved stocks, mixed with monolayer culture-expanded HUVECs at a 2:1 cell ratio, and a solution of type I collagen (final concentration: 1 μg/uL) prior to loading into a syringe for 3D printing. Cell/collagen mixtures were printed into 4.5% PEG, LLS microgels swollen with select plating/growth media. Two mm-diameter disc structures were generated, and microtissues were maintained under non-perfusion (no flow) conditions for 15 days. Cultures were fed by supplementing the microgel media with a small volume (0.3 mL) liquid growth media, which was exchanged every 4th day. To investigate the effects of perfusion, printed constructs were maintained in a custom-built 12-well perfusion system. For media exchange, under flow conditions, 1 mL of liquid media was perfused daily through the microgel media containing the printed microtissues via a feeding chamber and removed via an adjacent collection chamber.

Sample harvest and substrate addition: To assess metabolic function, a probe substrate cocktail specific for the activities of phase I and II enzymes (see Table 4 below) was incubated with the microtissues at day 15, by direct addition to the gel/well, or after transfer of the microtissues to Eppendorf tubes. Incubation was performed at 37° C., 5% CO₂ and sampling times were 0 (no addition), 30, 100 and 180 minutes following substrate addition.

TABLE 4
Probes, enzyme isoforms, concentrations, and metabolites.

| | | Incubation | |
|---|---|---|---|
| | | Concentration | |
| Probe Substrate | Isoform | (μM) | Metabolite |
| Phenacetin | CYP1A2 | 100 | Acetaminophen |
| Bupropion | CYP2B6 | 50 | Hydroxybupropion |
| Diclofenac | CYP2C9 | 25 | Hydroxydiclofenac |
| Dextromethorphan | CYP2D6 | 50 | Dextrorphan |
| Nifedipine | CYP3A4 | 15 | Oxidized Nifedipine |
| 7- | UGT1A1 | 10 | 7-Hydroxycoumarin |
| Hydroxycoumarin | | | Gluconoride |

Following incubation, microtissues were harvested in a ˜50 mL volume and mixed 1:4 with a reaction quench solution of 100% acetonitrile with 0.1% v/v formic acid and an internal standard cocktail. Samples were then frozen until evaluated by LC-MS analysis.

### Sample Preparation for LC-MS Analysis

Samples were thawed on wet ice. Samples were centrifuged in an Eppendorf table-top centrifuge (model 5425) at 3220 xg at 4° C. for 15 minutes to pellet precipitate. Following centrifugation, 100 μL of supernatant was transferred to a 96-well high recovery analysis plate. 100 μL of water was added to supernatant and briefly vortexed.

### LC-MS Analysis

LC-MS/MS analysis was performed on a Thermo Scientific (Waltham, Ma) LX-2 UPLC system with a Leap Autosampler interfaced to an Applied Biosystems/MSD Sciex (Framingham, MA) API-6500 mass spectrometer utilizing a turbo ion spray interface in both positive and negative modes. Separation was achieved using an Acquity UPLC HSS T3 column (1.8 μm, 2.1×50 mm) with a mobile phase consisting of 0.1% formic acid in water (solvent A) and 0.1% formic acid in acetonitrile (solvent B) at a flow rate of 0.75 mL/min. The LC gradient began at 5% B and held for 0.25 minutes then changed to 95% B over 1.5 min, held at 95% B for 0.42 minutes, and then returned to 5% B for 0.83 minutes. Analyte response was measured by multiple reaction monitoring (MRM) of transitions unique to each substrate's metabolite: m/z 312 to m/z 231 for hydroxydiclofenac, m/z 258 to m/z 157 for dextrorphan, m/z 256 to m/z 238 for hydroxybupropion, m/z 152 to m/z 110 for acetominophen, m/z 345.1 to m/z 284.3 for oxidized nifedipine, and m/z 337 to m/z 175 for 7-hydroxycoumarin glucuronide with m/z 329 to m/z 162.1 for labetalol internal standard and m/z 281.3 to m/z 193.1 for imipramine internal standard.

### Example 5

The work describes evaluation of different chemical formulations of microgels for the LLS media that will be synthesized and test their effects on microtissue biofabrication quality and functional liver microtissue function. The different chemical formulations will be tested on microtissues made from: pure hepatocytes; co-cultures of hepatocytes and stellate cells; co-cultures of hepatocytes, stellate cells, and cholangiocytes. A multi-layered cell printing approach will be taken initially to achieve tissues of millimeter scale in the lateral direction and less than 200 μm in thickness, requiring 5×10₄ to 2×10₅ cells per tissue, depending on tissue size and cell packing density.

### Overview and Principle Conclusions

As disclosed herein, copolymer microgels have been synthesized by two different routes. Charged microgels were prepared by precipitation polymerization to yield materials composed of acrylamide (pAAm), polyethylene glycol (PEG) and charged monomers including methacrylic acid (MAA), quaternized 2-(dimethylamino)ethyl methacrylate (qDMAEMA) and carboxybetaine methacrylate (CBMA). Charge-neutral microgels were synthesized from polyethylene glycol (PEG) via an inverse emulsion (water in kerosene) polymerization of PEG-acrylate and PEG-diacrylate to produce spherical microgels. Different formulations were tested and gel-size distributions were measured. Microgels from inverse emulsions are perfectly spherical, while microgels from precipitation polymerization are irregularly shaped. The perfectly spherical microgels pack onto the cell surface like a layer of hard spheres, making far fewer direct contact points than the irregularly shaped gels, that more uniformly blanket the cells. Early studies indicated that the charge-neutral PEG microgels always performed better than charged microgels. Since the PEG based microgel medium appears to have no chemically-based impact on any of the tissues tested in this project, efforts were begun to add advanced functionality to the microgels. Additionally sterilization protocols were developed in anticipation of these materials being commercialized or potentially used in tissue engineered medical products (TEMPs).

The initial material design was based on the idea that the microgels were to swell in the growth medium and largely serve as passive participants in the microtissue printing process, providing mechanical support and facilitating 3D fluid transport. The polymer mesh and pore space in the microgel medium is large enough to allow the unimpeded diffusion of large proteins and small molecules. However, chemical modification of the microgels provides opportunities for next-generation liquid-like solid printing media. It was reasoned that residual vinyl functionality within the microgels that inevitably results during radical-induced gelation could serve as reactive handles on which to conjugate a variety of moieties that can aid in the printing process, tissue viability, and in eventual tissue handling and manipulation. The initial modification approaches were designed to give microgels that included covalently-bound collagen-derived components of extracellular matrix that are essential for interaction and signaling between surrounding cells. A second modification procedure was designed to coat the microgels with a thermoresponsive polymer coating that could, when heated above ˜ 32° C., lead to microgel adhesion in the immediate vicinity of microtissues to create a cohesive shell that enables stabilization, harvesting, manipulation, storage, and shipping.

### 1. Functionalized Microgels

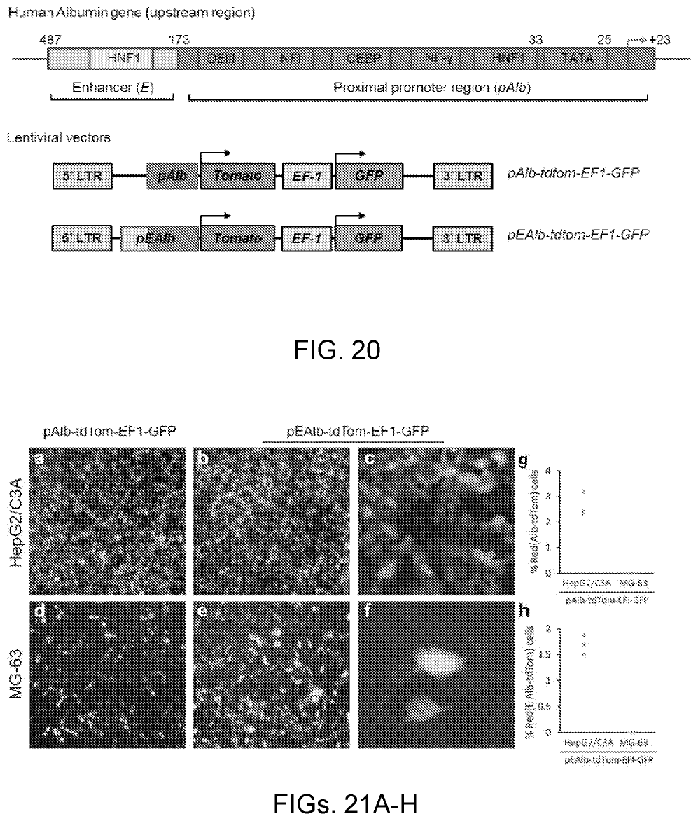

Two reaction strategies that could target the residual vinyl groups and achieve successful microgel functionalization were explored: (i) radical-induced thiol-ene reactions and (ii) thio-Michael addition. Radical-induced thiol-ene reactions proceed via chain reaction of the vinyl groups in the microgels with thiols to form stable thioether linkages (FIG. 28, top). Thio-Michael addition involves the same two reacting species but relies on a base-catalyzed nucleophilic 1,4-conjugate addition to α,β-unsaturated carbonyl compounds (FIG. 28, bottom).

To test the viability of this approach, a thiol-functional fluorescein was prepared that could be used in model studies to spectroscopically confirm successful targeting and reaction of the residual vinyl groups within the microgels.

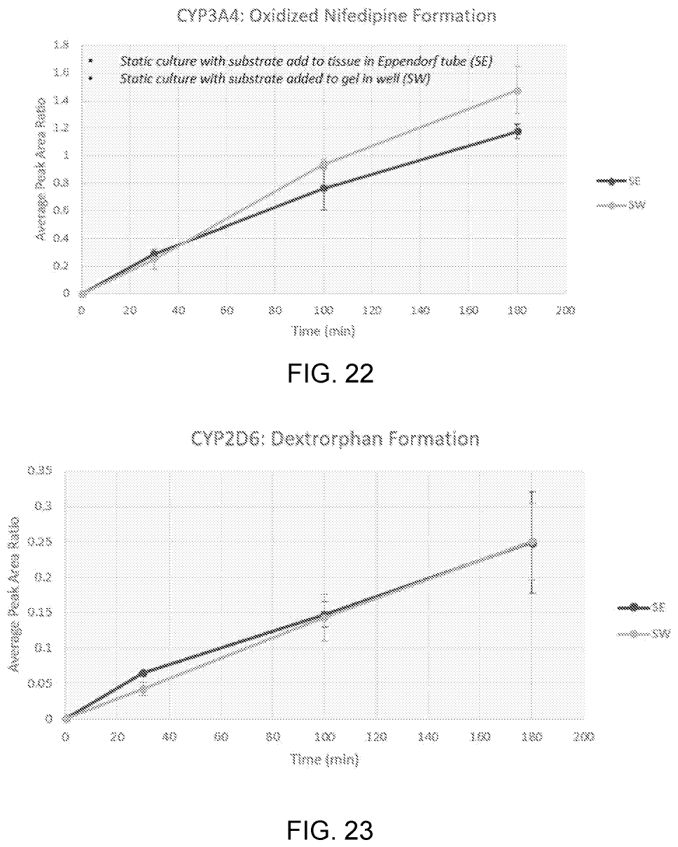

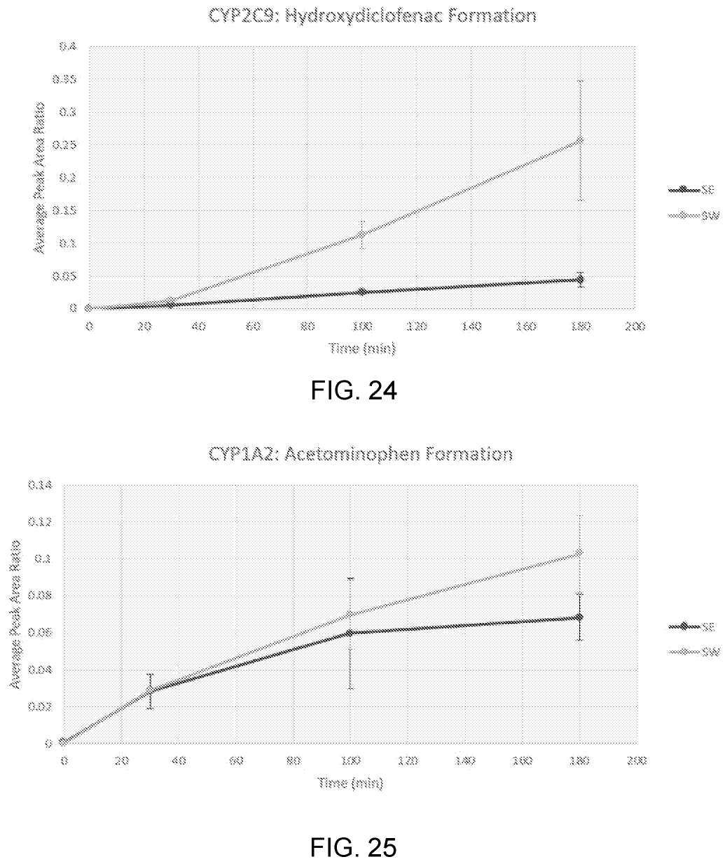

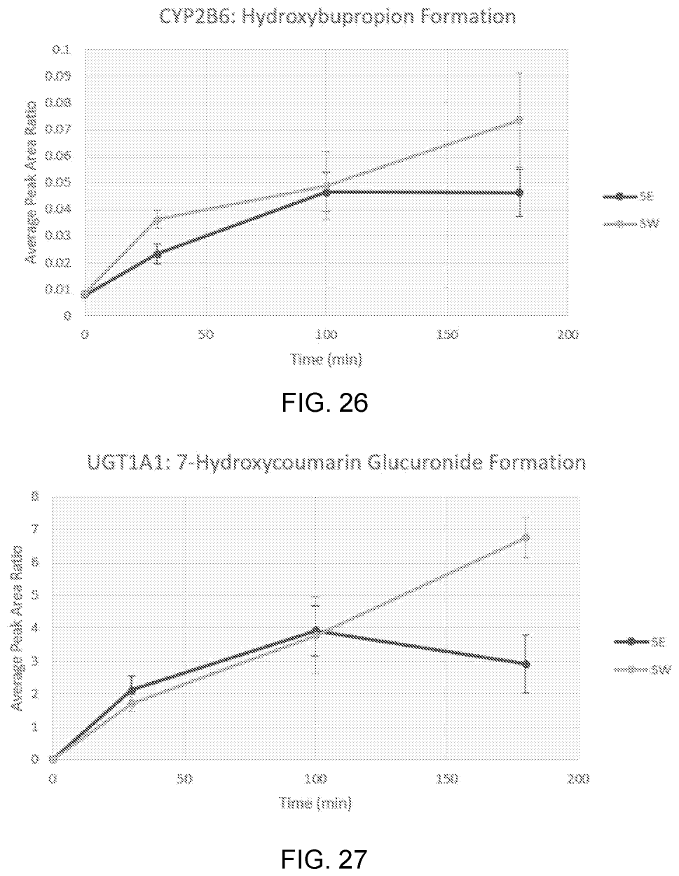

Fluorescein-thiol: Fluorescein-SH was prepared by an 1-ethyl-3-(3-dimethylaminopropyl) carbodiimide (EDC) coupling reaction between cystamine and fluorescein (FIG. 29). Fluorescence of the PEG gels after the reaction indicated successful targeting and functionalization of the vinyl groups via both thiol-Michael addition and the thiol-ene reaction pathways (FIG. 30). UV-vis spectrometry was used to confirm the successful conjugation of fluorescein thiol. The UV-vis absorbance was measured for the fluorescein thiol small molecule and compared to that of the conjugated PEG microgel. Extensive purification by dialysis against acetone for three days was carried out to ensure the removal of all unreacted dye. The UV absorbance at 550 nm for the conjugated PEG microgel verified the availability of residual vinyl groups to serve as synthetic handles for conjugation of other small molecules (FIG. 31).

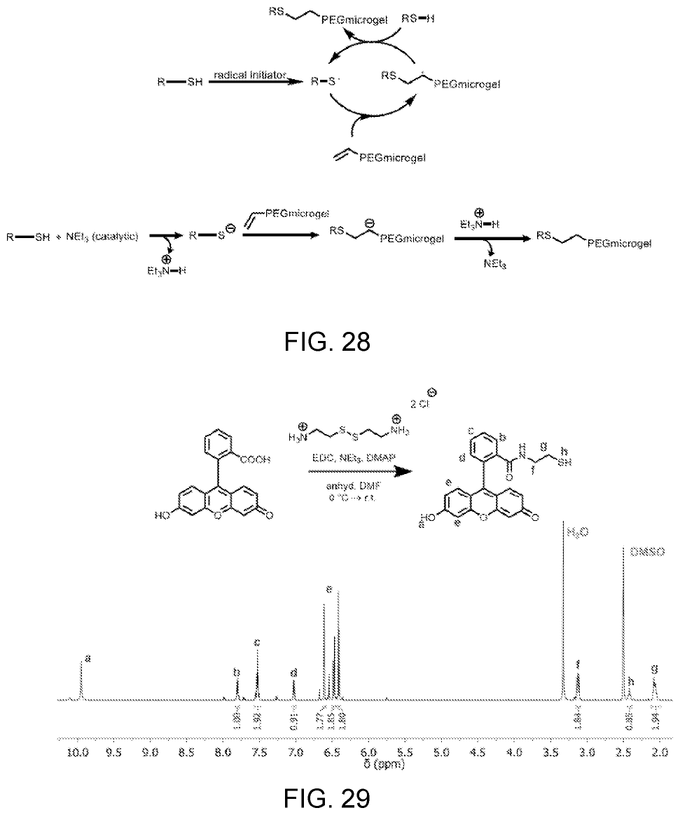

Gelatin-coated microgels. Being derived from the partial hydrolytic breakdown of collagen, gelatin is composed of relatively low molecular weight (<10 kDa) peptide components that promote cell adhesion and signaling. the thio-Michael addition route was employed to conjugate polythiolated gelatin to the PEG microgels. Briefly, PEG microgel and polythiolated gelatin were swollen in phosphate-buffered saline (PBS) and stirred at room temperature for 2 h (FIG. 32). The slightly basic pH of PBS sufficiently catalyzed the reaction.

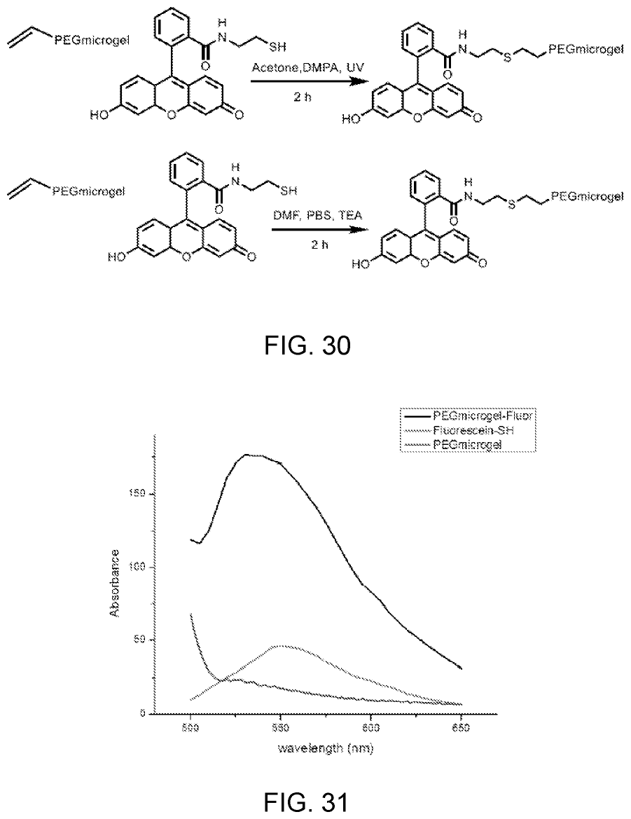

Following extensive purification by dialysis against acetone and water, ATR-FTIR analysis of the resulting lyophilized microgels suggested the residual vinyl groups of the PEG microgels had been largely consumed, which is consistent with their reaction with the polythiolated gelatin. The conjugated gels were purified by sequential dialysis in acetone and water, followed by lyophilization. Attenuated total reflectance-Fourier transform infrared (ATR-FTIR) spectroscopy of the resulting microgels suggested the residual vinyl groups of the PEG microgels had been consumed, evident by a decrease in the band around 2100 cm⁻¹ (FIG. 33). The formation of a C—S bond which should appear as a stretching band around 1300 cm⁻¹ was not observed, but this is likely due to the inherently low percent incorporation of conjugated species. Next steps include staining the gelatin-functionalized microgels with Siruis Red, an azo dye used to stain collagen and amyloid.

2. Thermoresponsive pNIPAM Coated Gels

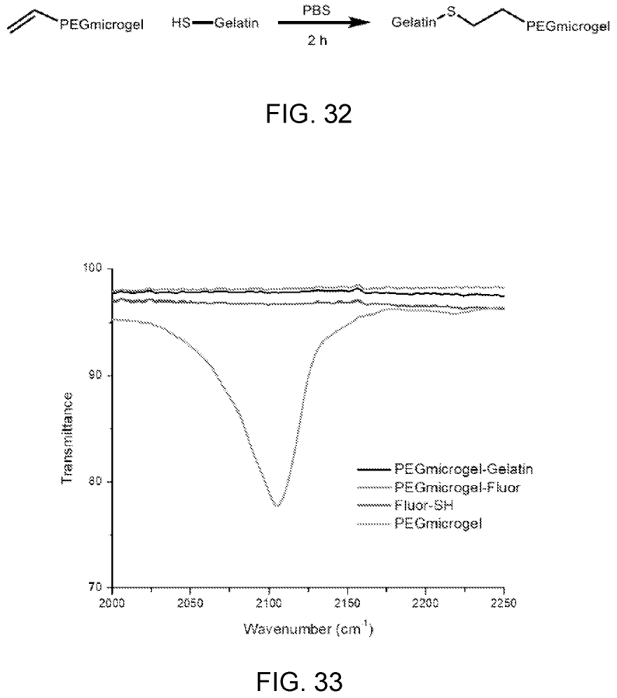

Poly(N-isopropylacrylamide) (PNIPAM) undergoes a hydrophilic-to-hydrophobic transition upon heating in an aqueous solution. Below approximately 32° C., PNIPAM is hydrophilic and water-soluble. Above this temperature, the polymer becomes hydrophobic and water-insoluble. It was reasoned that creating a thin coating of PNIPAM on the surface of the PEG microgels could lead to a liquid-like solid medium that could rigidify on heating due to inter-microgel aggregation via hydrophobic associations (FIG. 34). The hypothesis was that by printing a thin layer of thermoresponsive microgels around microtissues could lead to fusion of the microgels and reversible encapsulation of the microtissue. Such a process could support the microtissues after printing to enable harvesting, manipulation, storage, etc.

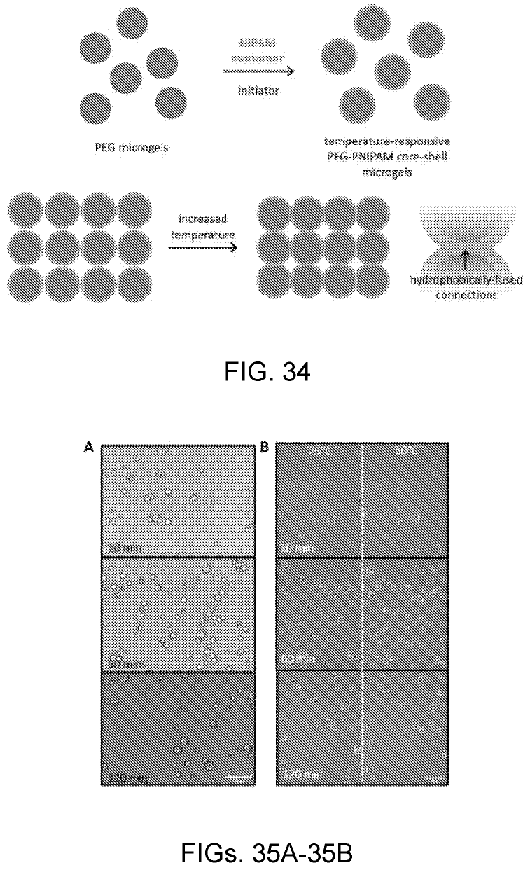

To achieve this coating, the microgels were allowed to absorb an aqueous solution of NIPAM monomer and a radical initiator for predefined periods of time before initiating the radical polymerization. It was reasoned that the polymerization process would lead to covalently integrated PNIPAM via cross-propagation of the newly initiated chains with the residual vinyl groups present in PEG microgels. Preliminary experiments suggest the PEG microgel decorated with PNIPAM did undergo dehydration and collapse of the shell layer, as demonstrated by a size reduction upon heating (FIGS. 35A-35B).

### 3. Sterilization of LLS Microgels for Microtissue Culture

To ensure sterility within the 3D printing and cell culture medium, heat sterilization of LLS microgels was performed following completion of the gel synthesis process. This additional step eliminates the microgels as a potential source of microbial contamination during microtissue fabrication and subsequent tissue culture. Autoclaving using a standard liquid cycle of 121° C. at 15 PSI was performed prior to swelling in hepatocyte-specific culture medium (see Appendix 2 for detailed protocol). After swelling, autoclaved gels were qualitatively indistinguishable from non-autoclaved gels, with no discernable effects on microsphere structure or flow properties.

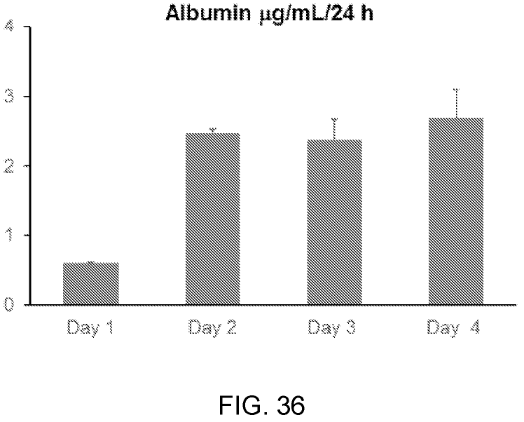

To verify that autoclaved gels can support hepatocyte functions, HepC3A cells were 3D printed into sterilized gels swollen with HepC3A-specific medium and maintained for 4 days. Media supernatants were collected each day and assayed for albumin synthesis by ELISA (Bethyl Laboratories). Daily albumin production (μg/mL) increased over the 4-day culture period (FIG. 36) indicating that gel sterilization had no detrimental effects on hepatocyte viability/synthetic function. Subsequent experiments were performed using heat sterilized LLS microgels and no incidences of microbial contamination were observed.

### Appendix 2: Protocol for LLS Microgel Sterilization

1. Autoclave gel swollen in PBS with the following liquid cycle settings:
   Temperature: 121° C.
   Pressure: 15 PSI
   Time: Adjust time according volume. (45 minutes for a bottle of 500 mL with 250 mL of gel, 30 minutes for 125 ml bottle with 60 mL of gel)
2. Allow gel to reach room temperature.
3. Aliquot gel in 50 mL sterile centrifuge tubes (maximum volume per tube: 40 mL).
4. Centrifuge gel for 30 minutes at 3600 rpm and 15° C.
5. Remove supernatant.
6. Add PBS (1× of gel volume) and then shake the tube vigorously.
7. Centrifuge gel for 30 min at 3600 rpm and 15° C.
8. Remove supernatant.
9. Repeat steps 5 to 7.
10. Add cell culture media (1× of gel volume) and then shake the tube vigorously.
11. Centrifuge gel 30 min at 3600 rpm and 15° C.
12. Remove supernatant.
13. Repeat steps 9 to 11.
14. Add ¼ of gel volume of cell culture media and mix.
15. Store at 4° C. for at least for 30 min prior to transferring to tissue culture plates for printing.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A planar cellular composition, comprising hepatocytes and endothelial cells in a matrix material, wherein the hepatocytes and endothelial cells are present in a ratio of about 2:1 to 1:1 hepatocytes:endothelial cells.

2. The planar cellular composition of claim 1, wherein the hepatocytes are primary cells.

3. The planar cellular composition of claim 1, wherein the endothelial cells are vascular endothelial cells.

4. The planar cellular composition of claim 1, wherein the hepatocytes and endothelial cells are present in a ratio of about 2:1 hepatocytes:endothelial cells.

5. The planar cellular composition of claim 1, wherein the matrix material comprises collagen.

6. The planar cellular composition of claim 1, wherein the planar cellular composition has at least one physical dimension between about 5 to 20 cells in thickness.

7. The planar cellular composition of claim 1, wherein the planar composition has a circular or oval shape.

8. The planar cellular composition of claim 6, wherein the circular or oval shape has a about 0.5 to 10 mm diameter, including about 1.5 to 2.5 mm in diameter.

9. The planar cellular composition of claim 1, wherein the endothelial cells form one or more sinusoidal structures that are not organized into a network.

10. A method of creating a planar cellular composition, comprising:
mixing hepatocytes, endothelial cells, and a matrix material in a cellular growth medium to form a cellular mixture, wherein the hepatocytes and endothelial cells are present in a ratio of about 15:10 to 20:15 hepatocytes:endothelial cells;
placing the cellular mixture in a support medium to form a planar cellular composition about 5 to 20 cells in thickness and about 0.5 to 10 mm diameter; and
culturing the cellular mixture under physiological growth conditions to produce the planar cellular composition.

11-21. (canceled)

22. A planar cellular composition produced according to the method of claim 9.

23. A tissue culture system, comprising:
the planar cellular composition of claim 1 embedded in a support medium; and
a bioreactor.

24. The system of claim 23, further comprising a cellular growth medium.

25. The system of claim 24, wherein the cellular growth medium comprises hepatocyte growth factor (HGF) but does not comprise vascular endothelial growth factor (VEGF).

26. The system of claim 23, wherein the bioreactor is a perfusion-enabled bioreactor.

27. The system of claim 23, wherein support medium comprises polymeric packed microgel particles.

28. The system of claim 23, wherein the support medium comprises poly-ethylene glycol (PEG) microparticles.

29. A method for screening candidate agents, comprising contacting the candidate agent to the planar cellular composition of claim 1 and evaluating one or more properties of the planar cellular composition.

30-32. (canceled)
